(19) United States
(12) Patent Application Publication
(10) Pub. No.: US 2015/0118728 A1
(43) Pub. Date: Apr. 30, 2015

(54) APPARATUS AND METHOD FOR SEPARATING A BIOLOGICAL ENTITY FROM A SAMPLE VOLUME

(71) Applicant: Agency for Science, Technology and Research, Singapore (SG)

(72) Inventors: Abdur Rub Abdur Rahman, Singapore (SG); Chandran Jegatha, Singapore (SG); Lakshmi Shankar, Singapore (SG); Chee Chung Wong, Singapore (SG)

(73) Assignee: AGENCY FOR SCIENCE, TECHNOLOGY AND RESEARCH, Singapore (SG)

(21) Appl. No.: 14/395,602

(22) PCT Filed: Apr. 19, 2013
(86) PCT No.: PCT/SG2013/000156

§ 371 Date: Oct. 20, 2014

(30) Foreign Application Priority Data
Apr. 20, 2012 (SG) SG201202943-5

(51) Int. Cl. C12N 13/00; B03C 1/30; B01L 3/00

(52) U.S. Cl. CPC C12N 13/00; B01L 3/502753; B03C 1/30; B01L 2200/06; B03C 2201/22; B01L 2300/0681
USPC 435/173.9; 422/534; 422/502; 422/535

(57) ABSTRACT

According to embodiments of the present invention, an apparatus for separating a biological entity from a sample volume is provided. The apparatus includes an input chamber including an inlet configured to receive the volume sample, and an outlet, at least one magnetic element adjacent a portion of the input chamber, the magnetic element configured to provide a magnetic field in a vicinity of the portion of the input chamber to trap at least some leukocytes from the sample volume, and a filter in fluid communication with the outlet, the filter configured to separate the biological entity. According to further embodiments of the present invention, a method for separating a biological entity from a sample volume is also provided.

### CROSS-REFERENCE TO RELATED APPLICATION

This application claims the benefit of priority of Singapore patent application No. 201202943-5, filed 20 Apr. 2012, the content of it being hereby incorporated by reference in its entirety for all purposes.

### TECHNICAL FIELD

Various embodiments relate to an apparatus and a method for separating a biological entity from a sample volume.

### BACKGROUND

Circulating tumor cells (CTCs) detection has emerged as a promising minimally invasive diagnostic and prognostic tool for patients with metastatic cancer. CTCs are prognostically critical, associated with clinical stage, disease recurrence, tumor metastasis, treatment response, and patient survival following therapy. It has been shown that patients with metastatic breast cancer having more than five CTCs per 7.5 mL of blood have a much lower survival rate than patients with fewer cells. However, the technical challenge remains in the capturing of CTCs along with the contamination of white blood cells (WBCs) and non-existence of efficient technology for their enrichment with high sensitivity and precision.

The ability to characterize CTCs requires not only the separation of target cells from a complex mixture but also the subsequent transport and manipulation of the isolated cells for further analysis. The number of WBCs per CTC is about 10⁶-10⁷, which makes an effective separation or enrichment step challenging yet crucial for further diagnosis. Enrichment at semi-quantitative levels is no more a requirement.

CTC detection includes three major constituents namely 1) enrichment of cells from blood, 2) detection of CTCs and 3) delivery/release of CTCs for downstream analysis. Current systems involve tedious protocols providing a number of sources of error.

The significance of CTCs for clinical cancer management has been widely recognized. In the last decade, CTC research has accelerated dramatically, as evidenced by the number of publications rising exponentially from less than 100 in the late 1980s to over 2800 in 2010. This is also accompanied by heightened commercial activity, with the existence of more than 100 companies worldwide catering to the market for CTC isolation products and services. Despite such rapid development and many efforts to develop technologies for tumor cell isolation, it has been widely noted by clinical-thought leaders that there is a lack of standardization and optimization of assays and that an optimal technology is still unavailable. The current understanding of clinical significance or the lack thereof of CTCs is biased on the technology used to isolate and detect these rare cells. For example, in 292 metastatic cancer patients, CTCs were not detected in 36% of the patients using the CellSearch® technique. Despite the presence of different conventional technologies, the lack of a standardized and optimized platform has been widely noted. Without an unbiased, standardized and optimized method for CTC isolation, CTCs may generate poor clinical interest.

Currently, CellSearch™ system from Veridex is an FDA-approved system for CTC level measurement where CTCs are identified through positive selection, and is currently used in most clinical trials to establish the utility of CTCs for clinical cancer management. This method uses ferrofluids loaded with an EpCAM antibody to capture CTCs, which are subsequently visualized by staining with a cocktail of antibodies against cytoplasmic epithelial cytokeratins. Staining for the leukocyte specific marker CD45 is used as a control to exclude contaminating leukocytes. Surprisingly, a significant number of cells appear to stain both for cytokeratin and CD45. Although this platform is the most standardized of any current technology and is now being tested for clinical applications, it suffers from relatively low sensitivity with a median yield of approximately one CTC per milliliter and does not separate EpCAM negative CTCs. Several conventional technologies have demonstrated substantially better performance than CellSearch®, which is currently used in many clinical trials to establish the validity of CTCs as a therapeutic efficacy marker. However, as Cellsearch and conventional technologies may fail to detect CTCs in some cancer patients, this raises the concern that if no CTCs are detected in a clinical sample (e.g. when a certain patient is deemed CTC-negative), is it due to the true absence of CTCs (i.e. true CTC-negative status of the patient) or a limitation of the technology used? Thus, this has led to calls for standardization and evaluation of technology platforms that may be optimally suited for CTC isolation and an urgent need to develop highly efficient and robust technologies for CTC isolation.

Furthermore, positive selection methods, as used in the CellSearch system, involve increased time consumption, e.g. in terms of processing, and poor sensitivity and specificity, with loss of detection of EpCAM negative CTCs.

The frequency of CTCs in blood is calculated to be approximately one per 10⁷-10⁹ blood cells, making highly sensitive tools requisite for their reliable capture and analysis. There are conventional systems available for detecting CTCs with some describing their molecular characterization. However, the specificity and reliability of some of these systems has been questioned, as many systems do not allow CTC visualization, and results have often been unreproducible.

The gold standard, which refers to the most optimal technology for CTC isolation, must first and foremost aim to isolate the maximum number of viable cells without relying on subjective (e.g. subjective to tumor heterogeneity and evolution) markers such as specific antigens or a specific size. Thus the most important performance parameter becomes the recovery efficiency. Secondly, various degrees of purity may be acceptable depending upon the contamination tolerance of downstream molecular analysis methods. Nevertheless, a method that targets the highest purity is considered to be superior. Thus, the second important performance parameter is the purity of isolated cells. This is followed by implementation parameters such as reproducibility, robustness, ease of use (e.g. automation), cost and test turnaround time (TAT). Due to the heterogeneous and mutagenic nature of tumor cells, which affect their biological and physical phenotypes, an ideal CTC platform should not depend on subjective markers such as antigen expression or physical parameters. An ideal CTC platform may be one that is based on a negative enrichment approach, which aims to remove all “normal” blood cells while retaining the “abnormal cells” for cytometric and molecular analyses.

The current technologies for CTC isolation platforms may be mainly categorized into two: a) antibody/antigen-based, which rely on a single or a combination of antibodies, and b) cell morphology-based, which rely on size and deformability of CTCs. It has been widely noted that antibody/antigen-based approaches (e.g. EpCAM-based method where Epithelial Cell Adhesion Molecule (EpCAM) is a common surface marker for antibody-based CTC isolation) may be susceptible to undercounting of CTCs due to cells undergoing Epithelial to Mesenchymal Transition (EMT) and hence not considered to be optimal. Size-based approaches may be susceptible to the overlapping of size and density between the CTCs and white blood cells (WBCs) and the heterogeneity in size and deformability within the tumor cell subpopulations, hence may be unable to overcome the recovery/purity trade-off. It has been reported that the tumor initiating sub-population of CTCs could easily pass through obstacles designed to trap cancer cells. Although physical filtration methods may be easy to implement, fast and relatively cost effective, the size-based CTC isolation technique suffers from an inherent recovery/purity trade-off and may possibly miss cells undergoing EMT, and therefore unable to fulfill the need for the most optimal technology for CTC isolation due to the inherent trade-off. A negative depletion approach, which avoids labeling the cells or defining their size, has been recommended as an optimal approach.

A negative depletion approach depletes “normal” cells, including erythrocytes (red blood cells), leukocytes, and platelets, and followed by depletion of white blood cells by immunomagnetic method, leaving behind the “abnormal cells” such as CTCs, which may then be identified by immunohistochemical staining or molecular analysis. In a typical negative enrichment approach, red blood cells (RBCs) may be eliminated by chemical lysis or ficoll gradient centrifugation followed by depletion of WBCs. This is followed by immunohistochemical staining or molecular analyses to identify the “abnormal cells”. Many currently developed negative depletion techniques involve multiple steps such as chemical RBC lysis and density gradient centrifugation that contribute to the risk of losing CTCs and adversely affect the cells of interest. It has been demonstrated using a negative enrichment method with a CTC isolation efficiency of 83% and 2.7 log₁₀ enrichment. This approach, however, involves multiple sample processing steps, including chemically RBC lysis, centrifugation, multiple cell washing and re-suspension, and isolation of peripheral blood mononuclear cells (PBMCs) using Ficoll density gradient separation. It was reported that RBC lysis and density gradient centrifugation steps can lead to 10% and 30% cell loss of spiked tumour cells respectively. Therefore, multiple sample processing steps are especially prone to compounding cell loss with each additional step, resulting in reducing overall efficiencies, thereby yielding much less than 100% overall CTC isolation efficiencies. Thus, a better implementation of a negative depletion or enrichment approach that avoids multiple sample handling steps or reduces the number sample handling steps, avoids centrifugation or use of chemicals, and usage of specialized instruments is needed.

With regard to available conventional systems, one of the systems employs an assay using a microfluidic device containing a silicon chip with microfabricated microposts, with immunity affinity based enrichment. However, it used anti-EpCAM for capture of cells, where the capture efficiency of the system is limited by variation in surface marker or antigen expression by CTCs, and that WBCs with larger sizes give error in counting. 2D or 3D filter based techniques have been used but requires samples to be partially fixed, incompatible for further live cell interrogations.

Other systems may employ the use of 1D channels/apertures for enrichment, 2D micro slots, circular filter, microcavity or may be based on immunomagnetic and immunofluorescent where the process is time consuming and subjective, and interpreting the immunofluorescent staining results requires a trained pathologist. A combination method of immuno-microparticles and density based separation has also been previously employed. However, in all the above mentioned techniques, the challenges that were not addressed are (1) sample storage, transfer and handling leading to cell loss which is detrimental to the overall cell yield (2) removal of WBCs that overlap with the size of the CTCs and (3) requirement of relatively simple equipment and providing superior observation capabilities, cell capture and release of captured cells at a faster rate for downstream analysis.

Other conventional methods include morphological separation where size or density is utilized to isolate CTCs from WBCs that overlap with the size of CTCs thus failing to capture the cancer cells that are as small as WBCs. These methods have significant barriers, including multiple procedural steps, substantial human intervention, high cost and importantly lack of high capture efficiency.

Thus, there is a need for the development of reliable, efficient platform to isolate, enrich and characterize CTCs in blood. The recent advent of the microfabrication technique may allow introduction of microchannel-based approaches for capture of these rare cells.

### SUMMARY

According to an embodiment, an apparatus for separating a biological entity from a sample volume is provided. The apparatus may include an input chamber including an inlet configured to receive the volume sample, and an outlet, at least one magnetic element adjacent a portion of the input chamber, the magnetic element configured to provide a magnetic field in a vicinity of the portion of the input chamber to trap at least some leukocytes from the sample volume, and a filter in fluid communication with the outlet, the filter configured to separate the biological entity.

According to an embodiment, a method for separating a biological entity from a sample volume is provided. The method may include supplying the sample volume to an input chamber, supplying a plurality of magnetic beads to the input chamber, the plurality of magnetic beads couplable to leukocyte specific biomarkers, trapping leukocytes from the sample volume that are coupled to the plurality of magnetic beads at a portion of the input chamber via at least one magnetic element, and filtering the sample volume by means of a filter for separating the biological entity.

According to an embodiment, a method for separating a biological entity from a sample volume is provided. The method may include supplying the sample volume to an input chamber, filtering the sample volume by means of a filter for trapping the biological entity and at least some of leukocytes, fetal cells or stem cells on the filter, supplying a plurality of magnetic beads couplable to leukocyte specific biomarkers to the filter, flowing the trapped contents of the filter into the input chamber, trapping leukocytes from the sample volume that are coupled to the plurality of magnetic beads at a portion of the input chamber via at least one magnetic element, and filtering the sample volume by means of the filter for separating the biological entity.

### BRIEF DESCRIPTION OF THE DRAWINGS

In the drawings, like reference characters generally refer to like parts throughout the different views. The drawings are not necessarily to scale, emphasis instead generally being placed upon illustrating the principles of the invention. In the following description, various embodiments of the invention are described with reference to the following drawings, in which:

FIG. 1A shows a schematic block diagram of an apparatus for separating a biological entity from a sample volume, according to various embodiments.

FIG. 1B shows a flow chart illustrating a method for separating a biological entity from a sample volume, according to various embodiments.

FIG. 1C shows a flow chart illustrating a method for separating a biological entity from a sample volume, according to various embodiments.

FIG. 2A shows a schematic cross sectional view of an input chamber of an apparatus for separating a biological entity from a sample volume, according to various embodiments.

FIG. 2B shows a schematic cross sectional view an apparatus for separating a biological entity from a sample volume, according to various embodiments.

FIG. 2C shows a schematic perspective view of a microfluidic device, according to various embodiments.

FIG. 2D shows a plot of Jurkat cell depletion using an apparatus of various embodiments.

FIGS. 3A to 3D show schematic cross sectional views of respective apparatuses for separating a biological entity from a sample volume, according to various embodiments.

FIGS. 4A and 4B show schematic cross sectional views of respective input chambers for an apparatus for separating a biological entity from a sample volume, according to various embodiments.

FIG. 5 shows a scanning electron microscope (SEM) image of a filter, according to various embodiments.

FIGS. 6A and 6B show schematic cross sectional views of respective filters, according to various embodiments.

FIGS. 6C and 6D show schematic cross sectional views of respective filters, according to various embodiments.

FIG. 7 shows a flow chart illustrating a method for separating a biological entity from a sample volume, according to various embodiments.

FIG. 8 shows a micro slit membrane, according to various embodiments.

FIG. 9A shows a cross sectional view of a microfluidic chip, while FIG. 9B shows a photograph image of the microfluidic chip of the embodiment of FIG. 9A, according to various embodiments.

FIG. 10 shows a plot of size distribution of cultured NCI-H1975 lung cancer cell line, according to various embodiments.

FIG. 11A shows a schematic perspective view of an apparatus for CTC isolation, while FIG. 11B shows a photograph image of an entire set-up for CTC isolation, according to various embodiments.

FIG. 12 shows a plot of WBC depletion (in percentage) as a function of the concentration of WBCs in original blood (in million per milliliter) from 25 blood samples.

FIG. 13 shows results of isolation and enumeration of spiking cancer cells. The nucleuses were stained with Hoechst (blue), the WBCs were stained by PE-labelled anti-CD45 (red) and the cancer cells were stained by FITC-labelled antibodies Pan-Cytokeratin (green).

FIGS. 14A and 14B show plots showing respectively the number of MCF-7 cancer cells and NCI-H1975 cancer cells, recovered and counted on the micro slit membrane as a function of number of cancer cells spiked into the 4-mL sample assay.

FIG. 14C shows a chart showing the average recovery efficiency of MCF7 and NCI-H1975 cancer cells. The error bars represent standard deviations among all experiments.

FIG. 15 shows fluorescence images under different filters for cells identification and classification, for Hoechst stains cell nucleus, PE-labelled anti-CD45 antibody stains CD45+ cells (WBCs), and FITC-labelled PanCK antibody stains PanCK+ cells (cancer cells).

FIG. 16 shows Hoechst-stained fluorescent images of nucleated cells captured by a micro slit membrane.

FIG. 17 shows Hoechst-stained, PE-labelled anti-CD45-stained and FITC-labelled antibodies Pan-Cytokeratin-stained fluorescent images relating to CTC isolation and enumeration from a NSCLC patient sample.

FIGS. 18A to 18C show photographs illustrating different views of a micro slit membrane, according to various embodiments.

FIG. 19 illustrates a CTC isolation system, according to various embodiments.

FIG. 20 shows a plot illustrating WBC depletion efficiency as a function of concentration of TAC in whole blood.

FIG. 21 shows a plot of results of WBC depletion (in percentage) obtained at different dilution factors under respective antibody loadings of 50 μL/mL and 100 μL/mL.

FIG. 22 shows the size ranges of a variety of cells within a blood sample compared to the sizes of tumor cells with the indication of pore size of current techniques.

FIGS. 23A and 23B show the summarized result of capture efficiency of tumor cells spiked into whole blood.

FIGS. 24A to 24C show images of MCF-7 cancer cells captured on micro slit membrane and imaged under fluorescence.

FIGS. 25A and 25B show fluorescent images (Hoechst) of nucleated cells captured by micro slit membrane with and without upstream WBC depletion, respectively.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Embodiments described in the context of one of the methods or devices are analogously valid for the other method or device. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element includes a reference to one or more of the features or elements.

In the context of various embodiments, the phrase “at least substantially” may include “exactly” and a reasonable variance.

In the context of various embodiments, the term “about” or “approximately” as applied to a numeric value encompasses the exact value and a reasonable variance.

As used herein, the term “and/or” includes any and all combinations of one or more of the associated listed items.

As used herein, the phrase of the form of “at least one of A or B” may include A or B or both A and B. Correspondingly, the phrase of the form of “at least one of A or B or C”, or including further listed items, may include any and all combinations of one or more of the associated listed items.

Various embodiments may relate to biosensor, for example relating to integrated microsystems for cell-based diagnostics, for example relating to circulating tumour cells (CTCs) in terms of diagnosis and therapy monitoring and/or endothelial progenitor cells (EPCs) for health monitoring.

Various embodiments may provide an automated rare cell enrichment system for highly efficient and cost effective enrichment. Various embodiments may provide an optimal and unbiased approach for tumor cell isolation.

Various embodiments may provide an automated meso/microfluidic integrated system for rare cell enrichment from biological samples, for example an automated rare cell (e.g. circulating tumor cells, CTC) enrichment system through white blood cell depletion from human whole blood. Various embodiments may provide enrichment of CTCs through a highly automated system (without or at least minimally affecting the classical phenotype). The automated system of various embodiments may enrich CTCs for downstream analysis.

Various embodiments may provide an approach of rare circulating tumor cell (CTC) enrichment using an automated, highly sensitive and specific meso/microfluidic system for isolation and enrichment of CTCs. The approach may include the combination of immune/immunomagnetic separation method for leukocytes or white blood cells (WBCs) depletion via negative selection and size based filtration for the removal of red blood cells (RBCs), yielding pure untethered CTCs from biological samples. The capture of white blood cells may be made possible using leukocyte specific biomarker(s) functionalized on an inner surface of, for example, a vacutainer/syringe barrel, and micro/nano particle based immunomagnetic separation. After which, the RBCs and the CTCs may flow through the microfluidic system which may include a filter membrane. The design of the pores on the filter membrane may allow most, if not all, the RBCs to flow through the filter membrane, paving the way for the effective enumeration of CTCs for downstream analysis. This may lead to a separation of pure CTCs without the loss of any EpCAM negative CTCs.

The fully automated system of various embodiments may include two steps wherein selective and specific enrichment of CTCs with/without EpCAM may be performed through a combination of immunoaffinity/immunomagnetic assay and filtration. Thus, this system as a whole may have the potential to provide highly specific CTCs for further molecular analysis in a fully automated manner.

Various embodiments may provide a set-up/apparatus to combine one or more white blood cell (leukocyte) depletion steps and one or more red blood cell depletion step in a single integrated flow path without the need to further handle (e.g. manually handle) the sample and/or the need for centrifugation.

In various embodiments, one of the WBC depletion steps may be or may include immuno-affinity based approach. In various embodiments, the RBC depletion step(s) may include or may be based on size and deformation, for example via filtration using a filter. The filter may be a one-dimensional, two-dimensional or three-dimensional filter, e.g. with ordered polygonal shapes or structures. The filter may be microfabricated, for example using lithography. In various embodiments, the WBC depletion step(s) and the RBC depletion step may be carried out in the same set-up/apparatus without the need to transfer the sample, e.g. blood, urine, or other bodily fluids.

In various embodiments, the set-up/apparatus may include a plurality of input reservoirs/chambers for receiving the sample and reagents and/or a plurality of output reservoirs/chambers, for example for receiving waste and reagents, such that the sample may be manipulated and/or processed without the need to take out or transfer the sample to a separate device.

In various embodiments, the sample may be flowed between an input reservoir/chamber and an output reservoir/chamber in a single direction or bi-directionally (including back flowing) to enhance the depletion of the WBCs and/or RBCs.

The apparatus or system of various embodiments may perform or provide one or more of the following functions: depletion of WBC (leukocytes), thereby enriching EpCAM negative CTCs; enhancement of both sensitivity and specificity of CTCs; easy availability of CTCs for downstream analysis.

Various embodiments may provide enrichment based on negative selection, and/or highly efficient WBC depletion from whole blood, and/or highly sensitive and specific cell enrichment (improved sensitivity and specificity), and/or an automated way of enrichment and detection in a single system which may reduce cell loss and/or requires no sample transfer, and/or reduced time consumption, e.g. in terms of processing, and/or a platform capability for high throughput sensing.

Various embodiments may be used for at least one of circulating tumour cell (CTC) detection, endothelial progenitor cell (EPC) detection, or maternal fetal cells (MFC) detection.

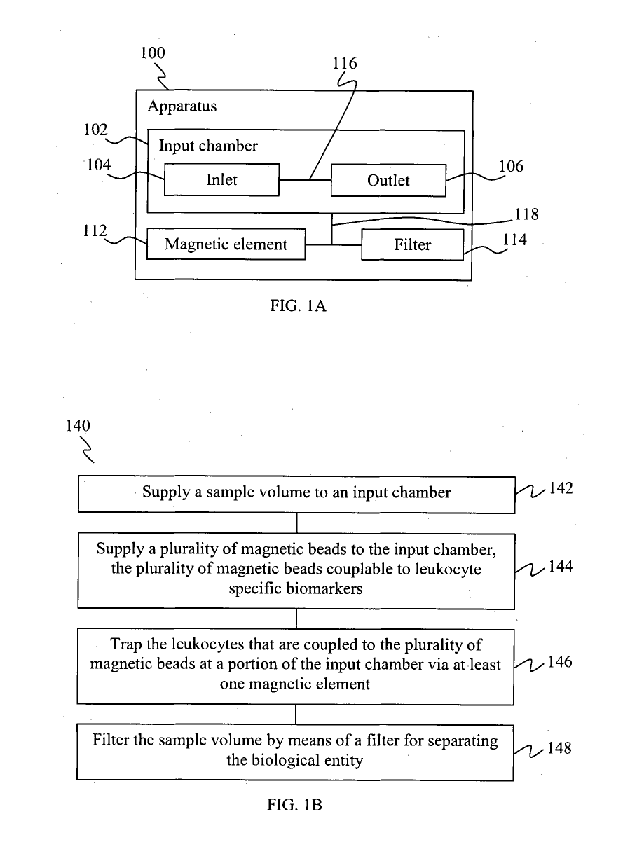

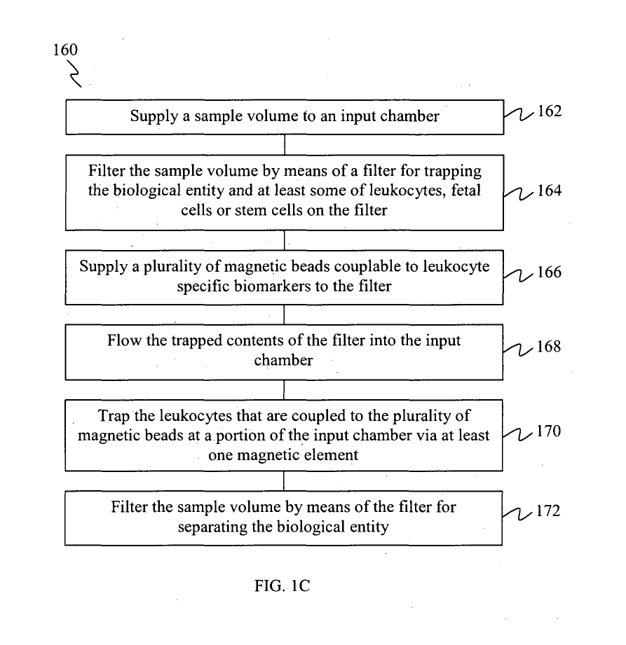

FIG. 1A shows a schematic block diagram of an apparatus 100 for separating a biological entity from a sample volume, according to various embodiments. The apparatus includes an input chamber 102 including an inlet 104 configured to receive the sample volume (e.g. blood, e.g. whole blood), and an outlet 106, at least one magnetic element 112 adjacent a portion of the input chamber 102, the magnetic element 112 configured to provide a magnetic field in a vicinity of the portion of the input chamber 102 to trap at least some leukocytes from the sample volume, and a filter 114 in fluid communication with the outlet 106, the filter 114 configured to separate or retain the biological entity. In FIG. 1A, the line represented as 116 is illustrated to show the relationship between the inlet 104 and the outlet 106 of the input chamber 102, which may include fluid communication with each other and/or mechanical coupling, while the line represented as 118 is illustrated to show the relationship among the input chamber 102, the magnetic element 112 and the filter 114, which may include fluid communication with each other and/or mechanical coupling.

In other words, the apparatus 100 may include an input chamber 102 having an inlet 104 and an outlet 106 that may be in fluid communication with each other. The apparatus 100 may further include at least one magnetic element 112 positioned adjacent a portion of the input chamber 102. This may trap leukocytes that may be present in the sample volume, within the input chamber 102. The leukocytes may be attached with magnetic beads. Therefore, by means of the at least one magnetic element 112 generating a magnetic field in a vicinity of the portion of the input chamber 102 for trapping at least some of the leukocytes from the sample volume, the sample volume that flows out of the outlet 106 may be at least substantially depleted of leukocytes as the leukocytes may remain within the input chamber 102. The apparatus 100 may further include a filter 114 in fluid communication with the outlet 106, where the filter 114 may retain the biological entity of interest, e.g. circulating tumour cells (CTCs), at the filter 114, while selectively allowing other materials, e.g. red blood cells (RBCs), to pass through the filter 114. Therefore, the filter 114 may serve to separate the biological entity from the sample volume.

In the context of various embodiments, the filter 114 may be configured to pass at least one of red blood cells (RBCs), platelets or at least some of the leukocytes.

In the context of various embodiments, the “biological entity” may include but not limited to a circulating tumour cell (CTC), a fetal cell or a stem cell.

In the context of various embodiments, the term “chamber” may include a well or a container. In the context of various embodiments, the term “chamber” may include a syringe or a vacutainer.

In the context of various embodiments, the magnetic element may include or may be a permanent magnet or an electromagnet. In the context of various embodiments, the magnetic element may be movable.

In the context of various embodiments, the sample volume may be a blood sample, urine, or other bodily fluids.

In various embodiments, the input chamber 102 may further include a layer including leukocyte specific biomarkers (e.g. antibodies) coated on at least a section of an inner wall of the input chamber 102, the leukocyte specific biomarkers configured to couple to leukocytes (or white blood cells, WBCs) from the sample volume. Therefore, leukocytes that may be present in the sample volume may be coupled or bound to the leukocyte specific biomarkers. In such embodiments, by means of the leukocyte specific biomarkers of the layer coated on at least a section of an inner wall of the input chamber 102, and the at least one magnetic element 112 generating a magnetic field in a vicinity of the portion of the input chamber 102 for trapping at least some of the leukocytes, the sample volume that flows out of the outlet 106 may be at least substantially depleted of leukocytes as the leukocytes may remain within the input chamber 102.

In the context of various embodiments, the term “leukocyte specific biomarkers” may mean biomarkers (e.g. antibodies) that may selectively couple or attach or bind with leukocytes. In the context of various embodiments, the leukocyte specific biomarkers may include but not limited to anti-CD45 specific antibodies.

In the context of various embodiments, the layer including leukocyte specific biomarkers (e.g. antibodies) that may be coated on at least a section of an inner wall of the input chamber 102 may further include an azide. The azide may be or may include but not limited to 4-azidoniline hydrochloride or amino azide or aldehydic azide or epoxy azide or aromatic-fluoro-nitro azide.

In various embodiments, the layer including leukocyte specific biomarkers may be coated throughout the inner wall of the input chamber 102. In various embodiments, the input chamber 102 may have a plurality of inner walls (e.g. sidewalls) and the layer including leukocyte specific biomarkers may be coated on at least a section of a respective inner wall or throughout a respective inner wall.

In various embodiments, the magnetic element 112 may be arranged to at least substantially surround the portion of the input chamber 102. In various embodiments, the magnetic element 112 may be arranged to at least substantially surround the input chamber 102 throughout the length of the input chamber 102.

In various embodiments, the apparatus 100 may further include a plurality of magnetic elements, e.g. 112, arranged along a length of the input chamber 102.

In various embodiments, the input chamber 102 and the filter 114 may form a closed pathway for the sample volume. In the context of various embodiments, the term “closed pathway” may mean a pathway that may not be accessible other than by way of the input chamber 102 and/or the filter 114. In other words, there are no intervening or intermediate structures or pathways that are coupled to or connected to any point of the closed pathway that may allow access to the sample volume.

In various embodiments, the filter 114 may be included in a microfluidic device. The filter 114 may be integrated in the microfluidic device. The microfluidic device may be at least substantially transparent. The microfluidic device may be made of a plastic or a polymer, e.g. polymethyl methacrylate (PMMA).

In various embodiments, the microfluidic device may include a piezoelectric substrate, e.g. made of a piezoelectric material including but not limited to lead titanate (PbTiO₃), lead zirconate titanate (PZT), lithium niobate (LiNbO₃), zinc oxide (ZnO) or polyvinylidene fluoride (PVDF).

In various embodiments, the outlet 106 of the input chamber 102 may be coupled to the filter 114 via at least one microchannel. The apparatus 100 may further include at least one further magnetic element arranged adjacent a portion of the microchannel, the magnetic element configured to provide a magnetic field in a vicinity of the portion of the microchannel.

In various embodiments, the input chamber 102 may further include a plurality of magnetic beads couplable to leukocyte specific biomarkers configured to couple to the leukocytes from the sample volume. The plurality of magnetic beads may be coated with leukocyte specific biomarkers.

In the context of various embodiments, the input chamber 102 may have a length of between about 10 mm and about 200 mm, for example between about 10 mm and about 100 mm, between about 10 mm and about 50 mm, between about 50 mm and about 200 mm, between about 50 mm and about 100 mm or between about 30 mm and about 80 mm.

In the context of various embodiments, the input chamber 102 may have a width or a diameter of between about 10 mm and about 100 mm, for example between about 10 mm and about 50 mm, between about 10 mm and about 30 mm, between about 50 mm and about 100 mm or between about 30 mm and about 50 mm.

In various embodiments, the filter 114 may include a single porous layer including a plurality of pores, each of the plurality of pores having a dimension between about 0.5 μm and about 30 μm, for example between about 0.5 μm and about 20 μm, between about 0.5 μm and about 10 μm, between about 0.5 μm and about 5 μm, between about 1 μm and about 10 μm, between about 5 μm and about 30 μm or between about 5 μm and about 10 μm.

In various embodiments, the filter 114 may include a first porous layer and a second porous layer arranged one over the other, wherein the first porous layer includes a plurality of first pores defined through the first porous layer, wherein the second porous layer includes a plurality of second pores defined through the second porous layer, and wherein one or more respective second pores may be arranged to at least substantially overlap with each respective first pore such that a respective opening defined between a perimeter of the each respective first pore and a perimeter of each of the one or more respective second pores may be smaller than a diameter of each first pore. Each second pore may have a diameter that is smaller than the diameter of each first pore. The one or more respective second pores may be arranged to be within the perimeter of each respective first pore.

In various embodiments, the filter 114 may include a plurality of first channels arranged in a first row, and a plurality of second channels arranged in a second row adjacent to the first row, wherein one or more respective second channels may be arranged to at least substantially overlap with each respective first channel such that a respective opening defined between an edge of the each respective first channel and an edge of each of the one or more respective second channels may be smaller than a width of each first channel. The filter 114 may further include a plurality of third channels arranged in a third row, wherein the second row may be arranged between the first row and the third row, and wherein one or more respective third channels may be arranged to at least substantially overlap with each respective second channel such that a respective opening defined between an edge of the each respective second channel and an edge of each of the one or more respective third channels may be smaller than a width of each second channel.

In various embodiments, the filter 114 may include a plurality of slits. Each slit may have a width of between about 4 μm and about 6 μm, for example between about 4 μm and about 5.5 μm or between about 5 μm and about 6 μm, and/or a length of between about 20 μm and about 50 μm, for example between about 20 μm and about 40 μm, between about 20 μm and about 30 μm or between about 40 μm and about 50 μm.

In various embodiments, the apparatus 100 may further include a valve in a fluid communication path between the outlet 106 of the input chamber 102 and the filter 114.

In various embodiments, the apparatus 100 may further include an output chamber in fluid communication with the filter 114, the output chamber configured to receive the sample volume after filtration through the filter 114.

In the context of various embodiments, the filter 114 may be a microfabricated filter.

In the context of various embodiments, the term “coupled” may include a direct coupling and/or an indirect coupling. For example, two devices being coupled to each other may mean that there is a direct coupling path between the two devices and/or there is an indirect coupling path between the two devices, e.g. via one or more intervening devices.

In the context of various embodiments, the term “connected” may include a direct connection and/or an indirect connection. For example, two devices being connected to each other may mean that there is a direct connection between the two devices and/or there is an indirect connection between the two devices, e.g. via one or more intervening devices.

Various embodiments may provide an apparatus for separating a biological entity from a sample volume, according to various embodiments. The apparatus may include an input chamber including an inlet configured to receive the sample volume (e.g. blood, e.g. whole blood), an outlet, and a layer including leukocyte specific biomarkers coated on at least a section of an inner wall of the input chamber, the leukocyte specific biomarkers configured to couple to leukocytes (or white blood cells, WBCs) from the sample volume, at least one magnetic element adjacent a portion of the input chamber, the magnetic element configured to provide a magnetic field in a vicinity of the portion of the input chamber to trap at least some of the leukocytes, and a filter in fluid communication with the outlet, the filter configured to separate or retain the biological entity.

FIG. 1B shows a flow chart 140 illustrating a method for separating a biological entity from a sample volume (e.g. a blood sample volume), according to various embodiments.

At 142, a sample volume is supplied to an input chamber.

At 144, a plurality of magnetic beads is supplied to the input chamber, the plurality of magnetic beads couplable to leukocyte specific biomarkers. In various embodiments, the plurality of magnetic beads may be coated with leukocyte specific biomarkers configured to couple to the leukocytes from the sample volume.

At 146, leukocytes from the sample volume that are coupled to the plurality of magnetic beads are trapped at a portion of the input chamber via at least one magnetic element.

At 148, the sample volume is filtered by means of a filter for separating or retaining the biological entity.

In various embodiments, the method may be performed based on the procedures at 142 to 148 in sequence.

In various embodiments of the method, at least a section of an inner wall of the input chamber may be coated with a layer including leukocyte specific biomarkers configured to couple to leukocytes from the sample volume. For example at 142, at least some leukocytes from the sample volume may be coupled to the leukocyte specific biomarkers of the layer.

In various embodiments, the sample volume may be filtered after removing the leukocytes from the sample volume by means of the layer including leukocyte specific biomarkers and/or the plurality of magnetic beads trapped by the magnetic element.

In various embodiments, the method may further include back flowing the sample volume through the filter. This may remove contents or materials trapped or retained by or at the filter, for example by back flowing the sample volume through the filter towards the input chamber.

FIG. 1C shows a flow chart 160 illustrating a method for separating a biological entity from a sample volume (e.g. a blood sample volume), according to various embodiments.

At 162, a sample volume is supplied to an input chamber.

At 164, the sample volume is filtered by means of a filter for trapping or retaining the biological entity and at least some of leukocytes, fetal cells or stem cells on the filter. This may mean that the biological entity and at least some of leukocytes, fetal cells or stem cells that may be present in the sample volume may be filtered by means of the filter.

At 166, a plurality of magnetic beads couplable to leukocyte specific biomarkers are supplied to the filter. The magnetic beads may be coupled to at least some of the leukocytes that may be trapped or retained by the filter.

At 168, the trapped or retained contents of the filter are flowed into the input chamber. This means that the contents or materials trapped or retained by or at the filter may be flowed, for example by back flowing through the filter, towards the input chamber.

At 170, leukocytes from the sample volume that are coupled to the plurality of magnetic beads are trapped at a portion of the input chamber via at least one magnetic element.

At 172, the sample volume is filtered by means of the filter for separating or retaining the biological entity.

In various embodiments, the method may be performed based on the procedures at 162 to 172 in sequence.

In various embodiments of the method, at least a section of an inner wall of the input chamber may be coated with a layer including leukocyte specific biomarkers configured to couple to leukocytes from the sample volume. For example at 162, at least some leukocytes from the sample volume may be coupled to the leukocyte specific biomarkers of the layer.

In various embodiments, the sample volume may be filtered after removing the leukocytes from the sample volume by means of the layer including leukocyte specific biomarkers at the input chamber and/or the plurality of magnetic beads trapped by the magnetic element.

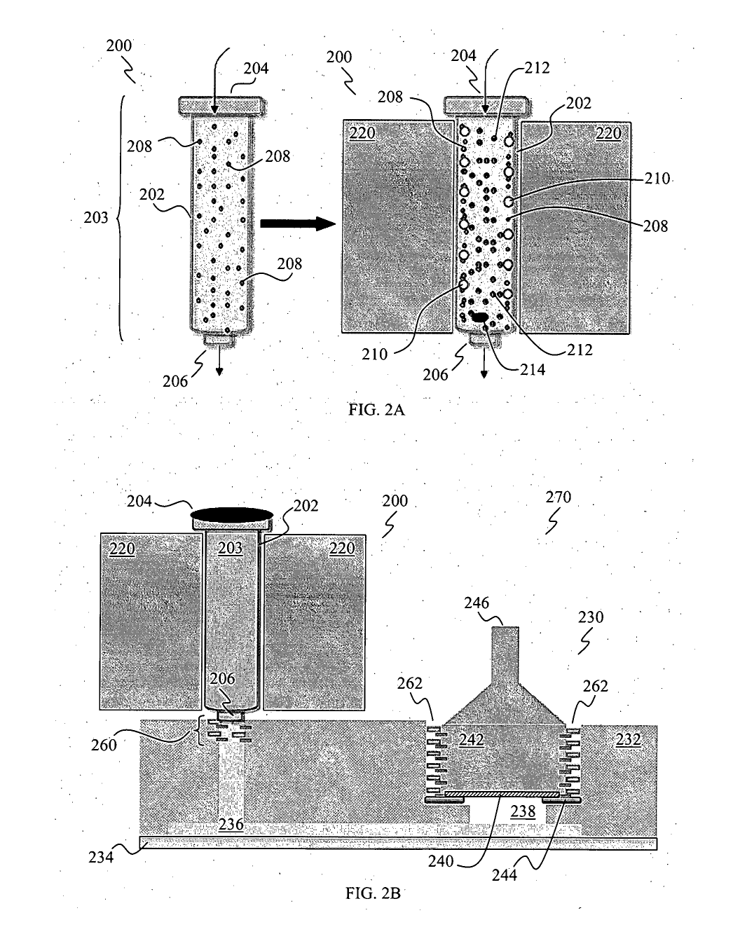

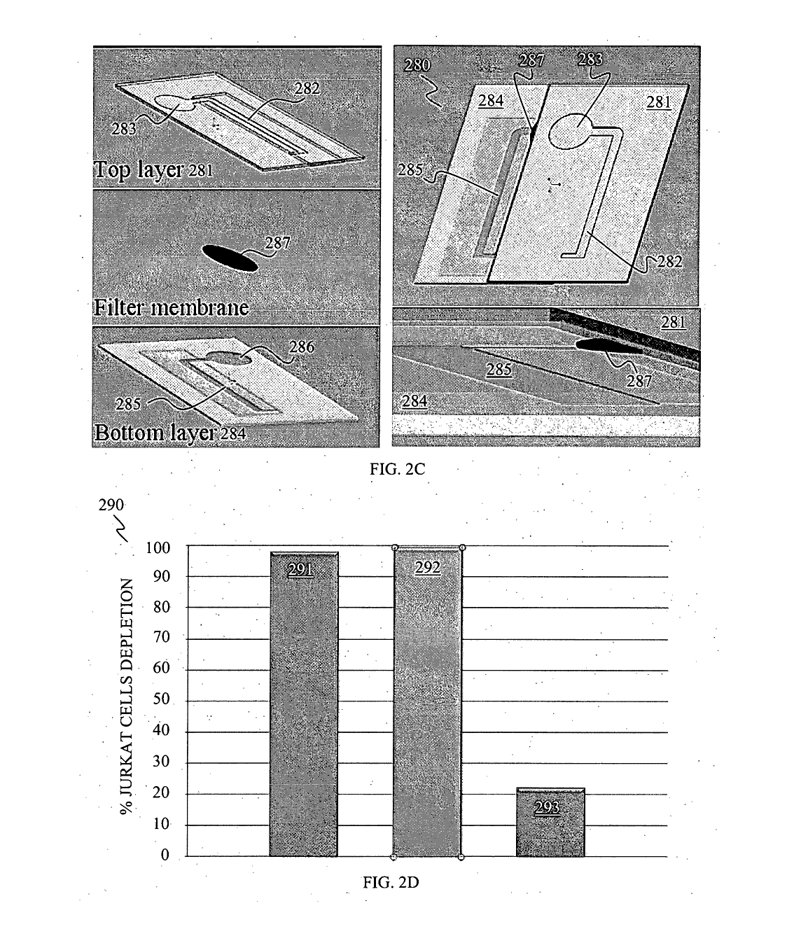

Various embodiments may provide an apparatus including cost-effective leukocyte specific biomarker coated syringe barrel coupled with leukocyte specific biomarker coated (micro/nano) magnetic beads for efficient WBC depletion. FIG. 2A shows a schematic cross sectional view of an input chamber 202 of an apparatus for separating a biological entity from a sample volume, according to various embodiments, illustrating a meso fluidic system 200 including a syringe barrel 202 with an inlet 204 through which a sample volume (e.g. biological sample) may be provided into the syringe barrel 202, and an outlet 206, through which, the sample volume may flow out of the syringe barrel 202, and micro/nano magnetic particles (e.g. as represented by 208 for some magnetic particles) coated with one or more leukocyte specific biomarkers. Thus, as a result, a functionalised syringe barrel 203 may be provided. While not shown, at least a section of an inner wall of the syringe barrel 202 may be coated with a layer of leukocyte specific biomarkers.

As shown in FIG. 2A, right figure, a sample volume including, among others, leukocytes or white blood cells (WBCs) (e.g. as represented by 210 for some WBCs), red blood cells (RBCs) (e.g. as represented by 212 for some RBCs), one or more CTCs (e.g. as represented by 214), and platelets (not shown), may be provided to the syringe barrel 202 through the inlet 204. Some of the WBCs 210 may bind to the micro/nano magnetic particles 208 which are coated with one or more leukocyte specific biomarkers, while some other WBCs 210 may bind to the leukocyte specific biomarkers coated on the inner wall, if present, of the syringe barrel 202. An external magnet 220 may be placed adjacent the syringe barrel 202, to provide a magnetic field, to attract the micro/nano magnetic particles 208 binded with the WBCs 210. Therefore, the WBCs 210 may be trapped or immobilised close to or adjacent the inner walls of the syringe barrel 202, while other constituents including RBCs 212 may remain free and mobile in the sample volume. Thereafter, the fluid sample mixture containing the RBCs 212 and CTCs 214 may pass through the outlet 206, for example to a microfluidic module.

FIG. 2B shows a schematic cross sectional view of an apparatus 270 for separating a biological entity from a sample volume, according to various embodiments. The apparatus 270 may be an integrated system for cell enrichment including the mesofluidic system 200 of the embodiment of FIG. 2A, for the removal of WBCs through immune/immunomagnetic separation and a microfluidic system 230 for the removal of RBCs through filtration, leaving behind pure untreated and viable CTCs at an increased speed for downstream molecular analysis. The microfluidic system 230 includes a microfluidic device or module 232 having a substrate (e.g. slide glass) 234. The functionalised syringe barrel 203 may be directly connected to the microfluidic device 232 using luer locks or connectors 260. For clarity purposes, the sample volume or micro/nano magnetic particles in the functionalised syringe barrel 203 are not shown.

The sample volume (e.g. biological sample, e.g. blood sample) may be provided to the apparatus 270 by being flowed through the inlet 204 of the syringe barrel 202. After removal of the WBCs, the fluid mixture containing RBCs and CTCs (e.g. WBC depleted blood sample) may flow through the outlet or exit 206 to the microfluidic device 232 for further CTC enrichment. The fluid mixture exiting the outlet 206 may flow through one or more microchannels 236 towards a microfluidic chamber 238. The microfluidic device 232 includes a filter or filter membrane 240 positioned or secured in a filter holder (e.g. open face filter holder) 242, which may be provided over a gasket 244 and within the microfluidic chamber 238. The filter membrane 240 may filter out all or at least a majority of the RBCs, leaving behind pure or predominantly CTCs. The filter holder 242 may be connected to the microfluidic device 232 using luer locks 262. As the fluid mixture flows through the filter 240, CTCs may be retained by the filter 240 within the microfluidic chamber 238 for further analysis while RBCs may pass through the filter 240 and through the outlet 246, for example to an output chamber or to waste.

In contrary to conventional approaches which identify the CTCs through positive selection, the fully automated system 270 removes the WBCs, thereby enriching the CTCs, through negative selection as shown in FIG. 2B. As a non-limiting example, in the apparatus 270, the WBCs may be isolated by mixing them with magnetic beads tagged with anti-CD45 specific antibodies, followed by application of a magnetic field. Also, WBCs may be captured by the antibody-coated inner surface of the syringe barrel 202. Both the bottom and top layer of the microfluidic device 232 may be maintained at least substantially transparent, allowing the feasibility of optical detection of captured CTCs as shown in in FIG. 2B. The filter membrane 240 may have pores designed in such a way that the RBCs may squeeze through the pores, while retaining CTCs, including EpCAM negative CTCs, on the filter membrane 240. Thus, this technology may eliminate or at least minimise the loss of cells due to sample transfer.

In various embodiments, the flow of the sample volume may be monitored through pressure pumps to avoid or minimise the destruction of cells. The use of an immune/immunomagnetic separation system 200 may help to remove a major portion of WBCs from blood before it reaches the microfluidic system 230. Pre-enriched blood sample may then enter into an integrated microfluidic system 230 for the removal of RBCs via filtration to obtain only or predominantly CTCs. The pre-enrichment and integrated steps may be fully automated as shown in FIG. 2B. The automated system 270 has the potential to provide highly efficient enrichment for further analysis.

In various embodiments, the combination of leukocyte specific biomarker coated on the inner surface of the syringe barrel and on magnetic micro/nanoparticles added within the syringe barrel 202 in a reproducible/consistent manner may be of importance in terms of various parameters such as concentration, incubation time, temperature, vortexing etc to deplete WBCs and enrich untethered CTCs from human whole blood/biological samples.

In various embodiments, the fabrication process may involve forming a meso/microfluidic system 270 for sample flow and integration of filter membrane 240 within the microfluidic platform 230. The microfluidic chamber 238 may be defined using soft lithography and micromachining methods. The complete assembly of the microfabricated device may be achieved through plasma bonding technique. The filter membrane 240 may be integrated within the microfluidic system 230 and used with pressure monitoring systems. The fully integrated automated system 270, as shown in FIG. 2B, for CTC enrichment may have the potential to provide fully automated, easy, less labour intensive and cost effective technique for new generation cancer diagnostic tools.

FIG. 2C shows a schematic perspective view of a microfluidic device 280, according to various embodiments. FIG. 2C, left figures illustrate the individual components of the microfluidic device 280, while FIG. 2C, right figures illustrate the assembled microfluidic device 280. The microfluidic device 280 includes a top layer 281 including a microchannel 282 and a circular portion or well 283, and a bottom layer 284 including a microchannel 285 including a circular portion or well 286, where the circular portions 283, 286, define a microfluidic chamber, and within which a filter membrane 287 may be arranged, when assembled. The top layer 281 and the bottom layer 284 may be made of polymethyl methacrylate (PMMA). The filter membrane 287 may be made of Parylene-C.

FIG. 2D shows a plot 290 of Jurkat cell depletion using an apparatus of various embodiments, for Jurkat cells contained in antibody (Ab)-coated tubes as the input chamber. The plot 290 shows the results 291 for Jurkat cells in Ab-coated tubes, provided with magnetic beads and with vortex, results 292 for Jurkat cells in Ab-coated tubes, provided with magnetic beads, and positive control results 293 for Jurkat cells in Ab-coated tubes. As can be seen from the results 291 and 292, there is an improvement in Jurkat cells depletion as compared to the positive control results 293.

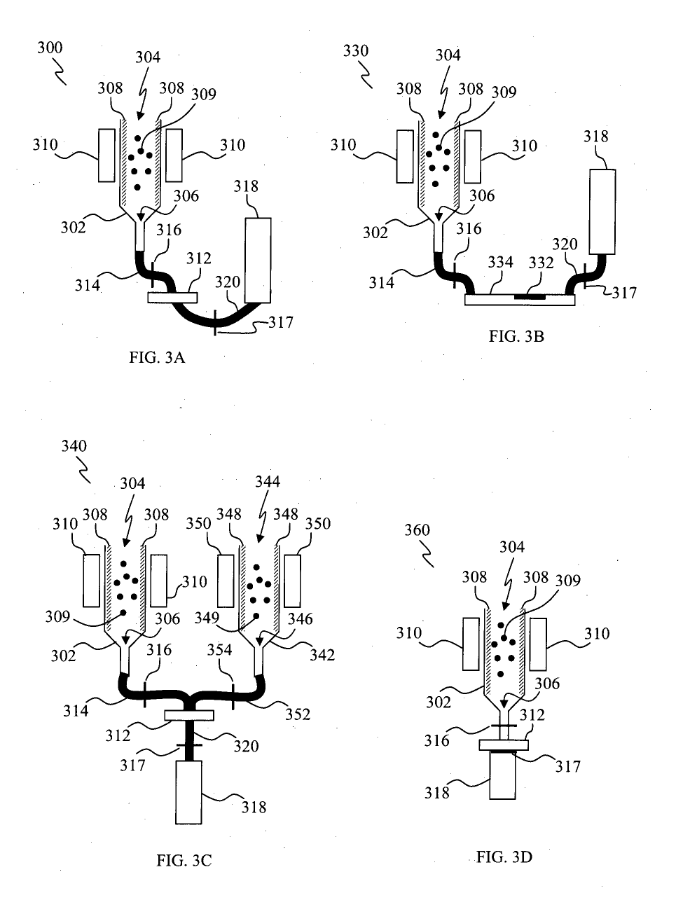

FIG. 3A shows an apparatus 300 for separating a biological entity from a sample volume (e.g. a blood sample volume). The apparatus 300 includes an input chamber 302, the input chamber 302 including an inlet 304 configured to receive the volume sample, an outlet 306 and, optionally, a layer 308 including leukocyte specific biomarkers coated on at least a section of an inner wall of the input chamber 302, the leukocyte specific biomarkers configured to couple to leukocytes (white blood cells) from the sample volume, at least one magnetic element 310 adjacent a portion of the input chamber 302, the magnetic element 310 configured to provide a magnetic field in a vicinity of the portion of the input chamber 302 to trap at least some of the leukocytes from the sample volume, and a filter (or filter membrane) 312 in fluid communication with the outlet 306, the filter 312 configured to separate and retain the biological entity, whilst allowing passage of most erythrocytes, some leukocytes and most platelets to the waste or output chamber. In various embodiments, the layer 308 may at least substantially surround the section of the inner wall of the input chamber 302.

In various embodiments, a layer including leukocyte specific biomarkers (e.g. similar to layer 308) may also be coated on at least a section of an inner wall of any connecting channel or path, for example a tubing interconnection or a microchannel, between the outlet 306 and the filter 312, leading up to the filter 312.

In various embodiments, it should be appreciated that the input chamber 302 may include a plurality of inlets and/or a plurality of outlets.

In various embodiments, the input chamber 302 may be provided or manufactured such that the internal surface of the input chamber 302, which may be in contact with a sample, may be roughened (i.e. provided with a rough surface) to produce a large surface area, thus providing more capture surface for leukocytes.

The preparation for the layer 308 will now be described by way of the following non-limiting example. A photoreactive substance may be physically deposited on at least a section of an inner wall or surface of the input chamber 302 through Azido chemistry. The reaction between the inner surfaces (e.g. polymer surfaces) of the input chamber 302 may occur steadily under ultraviolet (UV) exposure (e.g. at about 220 nm). After which glutaraldehyde (GAD) and sodium cyanoborohydride may be used as further reagents to further enhance the functionalization by providing anchor sites for the capture of leukocyte specific biomarkers.

After functionalizing the surface to capture leukocyte specific biomarkers, the biomarkers may be deposited in liquid phase by diluting it in liquid and incubating in the input chamber 302 as per the following surface treatment protocol for the treatment of the inner wall of the input chamber 302.

The surface treatment protocol may facilitate binding between a substrate (e.g. a plastic substrate) and antibody, hence capturing (or trapping) blood cells through antibody-antigen specific binding between proteins on blood cells. The procedure for the surface treatment may be as follows:
1. Mix about 0.5-1 mg of 4-azidoniline hydrochloride with ethanol.
2. Treat the surface (e.g. plastic surface) of the substrate with the azido-solution. This step should be operated in a substantially dark environment to minimize the unwanted reactions due to exposure of light.
3. Cure the sample for about 60 min at room temperature with gentle shaking.
4. Treat the surface under UV transilluminator (e.g. at about 220 nm) for about 10-30 min.
5. Wash the surface with ethanol, subsequently with de-ionised (DI) water.
6. Incubate the surface with 2% Glutaraldehyde for about 30 min.

7. Wash the surface with phosphate buffered saline (PBS).
8. Incubate the surface with leukocyte specific biomarker for about 30 min.
9. Wash the sample with PBS again.

Based on the surface treatment protocol, 4-azidoniline hydrochloride may be physically deposited on the surface (e.g. polymer/plastic surface) through evaporation of ethanol. Under UV light, the chemical reaction between the polymer and the chemical substance may occur steadily. Glutaraldehyde (GAD) may be used to enhance functionalization to provide more anchor-sites (chemical groups) for binding of antibody.

In further embodiments, the input chamber 302 may also be coated via spray coating of the leukocyte specific biomarkers after surface activation, using spray coating means.

In various embodiments, the apparatus 300 or the input chamber 302 may further include a plurality of magnetic beads, as represented by 309 for one bead, couplable to or configured to couple to leukocyte specific biomarkers, which in turn are configured to couple to the leukocytes from the sample volume. For example, antibody may be conjugated to the cells (e.g. leukocytes) by mixing the antibody solution with the sample volume, and then the magnetic beads may be conjugated to the antibody. When the magnetic element 310 is arranged to be adjacent a portion of the input chamber 302, the magnetic beads 309 may be trapped by the magnetic field induced by the magnetic element 310. In various embodiments, the plurality of magnetic beads 309 may be coated with leukocyte specific biomarkers configured to couple to the leukocytes from the sample volume.

In various embodiments, a least a portion of a plurality of magnetic beads (e.g. 309) may be coated with or couplable to biomarkers specific to non-targeted cells (e.g. red blood cells), other than leukocytes.

In various embodiments, the filter 312 may be provided in a filter holder. The filter holder may be a commercial filter holder. In various embodiments, the filter 312 may be a microfabricated filter. The microfabricated filter may be manufactured to fit inside commercial filter holders (e.g. 13 mm or 25 mm filter holders).

As shown in FIG. 3A, the outlet 306 of the input chamber 302 may be connected to or in fluid communication with the filter 312 via a tubing interconnection 314. A valve 316 may be provided to control the flow of the sample volume between the input chamber 302 and the filter 312. In other words, a valve (e.g. 316) may be provided in or along a fluid communication path between the outlet 306 of the input chamber 302 and the filter 312.

The apparatus 300 may further include an output chamber 318 in fluid communication with the filter 312, the output chamber 318 configured to receive the sample volume after filtration through the filter 312. As shown in FIG. 3A, the filter 312 may be connected to or in fluid communication with the output chamber 318 via a tubing interconnection 320. In addition or alternative to valve 316, a valve 317 may be provided to control the flow of the sample volume between the filter 312 and the output chamber 318.

It should be appreciated that the two rectangular blocks represented by 310 for the at least one magnetic element may be two separate magnetic elements or may be a continuous magnetic element. In various embodiments, the magnetic element 310 may be arranged to at least substantially surround the portion of the input chamber 302.

A magnetic element may also be placed along any section of the path between the outlet 306 of the input chamber 302 and the filter 312, for example along at least a section of the tubing interconnection 314.

In various embodiments, the input chamber 302 and the filter 312 may form a closed pathway for the sample volume. In addition, the input chamber 302 (which may include one or more inlets (e.g. 304) and/or one or more outlets (e.g. 306)), the filter 312 and the output chamber 318 may also form a closed pathway. In the context of various embodiments, the term “closed pathway” means a pathway that may not be accessible other than by way of the input chamber 302 and/or the filter 312 and/or the output chamber 318. In other words, there are no intervening or intermediate structures or pathways that are coupled to or connected to any point of the closed pathway that may allow access to the sample volume.

In various embodiments, the sample volume may be flowed from the input chamber 302 to the filter 312. In addition, a backflow may be provided to flow the sample volume from the filter 312 to the input chamber 302. This may enhance the depletion of leukocytes from the sample volume as the sample volume passes through the input chamber 302 twice or more times as a result of the backflow or repeated backflows, so as to be captured or coupled to the leukocyte specific biomarkers of the layer 308 and/or of the magnetic beads 309.

In one embodiment, diluted whole blood may first be flowed through to isolate non-targeted leukocytes and targeted rare cells on the surface of the filter 312, whilst allowing passage of erythrocytes (red blood cells), some leukocytes (white blood cells) and platelets, as well as blood plasma through. In a subsequent step, isolated cells on the filter 312 may be back flowed into the input chamber 302. Thereafter, magnetic labeling reagents may be added to the input chamber 302. After appropriate incubation, the sample may be passed through the filter 312 again. This method has the benefit of requiring substantially less antibody, which is very expensive, as opposed to labeling the leukocytes in the entire whole blood sample. This method also has the benefit of better labeling efficiency, as the labeling occurs in a less biologically complicated media than whole blood.

In the context of various embodiments, the magnetic element 310 may be movable, allowing it to be positioned adjacent a portion of the input chamber 302 when desired and away from the input chamber 302 when desired.

In the context of various embodiments, the magnetic element 310 may be an electromagnet, which may be switched on and off as desired.

FIG. 3B shows an apparatus 330 for separating a biological entity from a sample volume (e.g. a blood sample volume), which may be similar to the apparatus 300 and as described in the context of the apparatus 300, except that the filter (e.g. a microfabricated filter) 312 of the apparatus 300 is comprised in a microfluidic device 334 The outlet 306 of the input chamber 302 may be coupled to the microfabricated filter 332 via the tubing interconnection 314 and at least one microchannel (not shown) on the microfluidic device 334. Furthermore, the apparatus 330 may include at least one magnetic element (not shown) adjacent a portion of the microchannel, the magnetic element configured to provide a magnetic field in a vicinity of the portion of the microchannel. In addition, the output chamber 318 may be coupled to the microfabricated filter 332 via the tubing interconnection 320 and at least one other microchannel (not shown) on the microfluidic device 334. In various embodiments, the microfluidic device 334 may be at least substantially transparent, for example to allow optical characterization. In various embodiments, the microfluidic device 334 may have a piezoelectric substrate.

While the tubing interconnections 314, 320 are illustrated in FIG. 3B for coupling of the outlet 306 of the input chamber 302 and the output chamber 318 respectively to the microfluidic device 334, it should be appreciated that the outlet 306 of the input chamber 302 and/or the output chamber 318 may be connected directly to the microfluidic device 334, for example by means of luer locks or connections via valves.

FIG. 3C shows an apparatus 340 for separating a biological entity from a sample volume (e.g. a blood sample volume), which may be similar to the apparatus 300 and as described in the context of the apparatus 300. The apparatus 340 further includes a second input chamber 342, the input chamber 342 including an inlet 344 configured to receive the volume sample, an outlet 346 and optionally a layer 348 including leukocyte specific biomarkers coated on at least a section of an inner wall of the input chamber 342, the leukocyte specific biomarkers configured to couple to leukocytes (white blood cells) from the sample volume, at least one magnetic element 350 adjacent a portion of the input chamber 342, the magnetic element 350 configured to provide a magnetic field in a vicinity of the portion of the input chamber 342 to trap at least some of the leukocytes. The filter 312 is in fluid communication with the outlet 346.

In various embodiments, the input chamber 342 may further include a plurality of magnetic beads, as represented by 349 for one bead, couplable to or configured to couple to leukocyte specific biomarkers, which in turn are configured to couple to the leukocytes from the sample volume. When the magnetic element 350 is arranged to be adjacent a portion of the input chamber 342, the magnetic beads 349 may be trapped by the magnetic field induced by the magnetic element 350. In various embodiments, the plurality of magnetic beads 349 may be coated with leukocyte specific biomarkers configured to couple to the leukocytes from the sample volume.

In various embodiments, a least a portion of a plurality of magnetic beads (e.g. 349) may be coated with or couplable to biomarkers specific to non-targeted cells (e.g. red blood cells), other than leukocytes.

As shown in FIG. 3C, the outlet 346 of the input chamber 342 may be connected to or in fluid communication with the filter 312 via a tubing interconnection 352. A valve 354 may be provided to control the flow of the sample volume between the input chamber 342 and the filter 312. In other words, a valve (e.g. 354) may be provided in or along a fluid communication path between the input chamber 342 and the filter 312.

Therefore, the apparatus 340 may include two input chambers, for example a first input chamber 302 and a second input chamber 342, which may receive the sample volume. This may allow simultaneous or consecutive processing of the sample volumes contained in the first input chamber 302 and the second input chamber 342.

In further embodiments, the sample volume may be provided in the first input chamber 302, which is then flowed to the filter 312. A backflow may then be provided through the filter 312 and the sample volume may then be flowed to the second input chamber 342 or the first input chamber 302.

In addition or alternative to valves 316 and/or 354, a valve 317 may be provided to control the flow of the sample volume between the filter 312 and the output chamber 318.

While the filter (e.g. a microfabricated filter) 312 is illustrated in FIG. 3C, the microfabricated filter 332 comprised in a microfluidic device 334 as described in the context of the apparatus 330 may instead be used.

FIG. 3D shows an apparatus 360 for separating a biological entity from a sample volume (e.g. a blood sample volume), which may be similar to the apparatus 300 and as described in the context of the apparatus 300, except that the outlet 306 of the input chamber 302 is connected directly to one side of the filter 312, for example by means of luer locks or connections, while the output chamber 318 is connected directly to another side of the filter 312, for example by means of luer locks or connections.

In various embodiments, the apparatus 360 includes a valve 316 to control the flow of the sample volume between the input chamber 302 and the filter 312, and/or a valve 317 to control the flow of the sample volume between the filter 312 and the output chamber 318.

While the filter 312 is illustrated in FIG. 3D, the microfabricated filter 332 comprised in a microfluidic device 334 as described in the context of the apparatus 330 may instead be used.

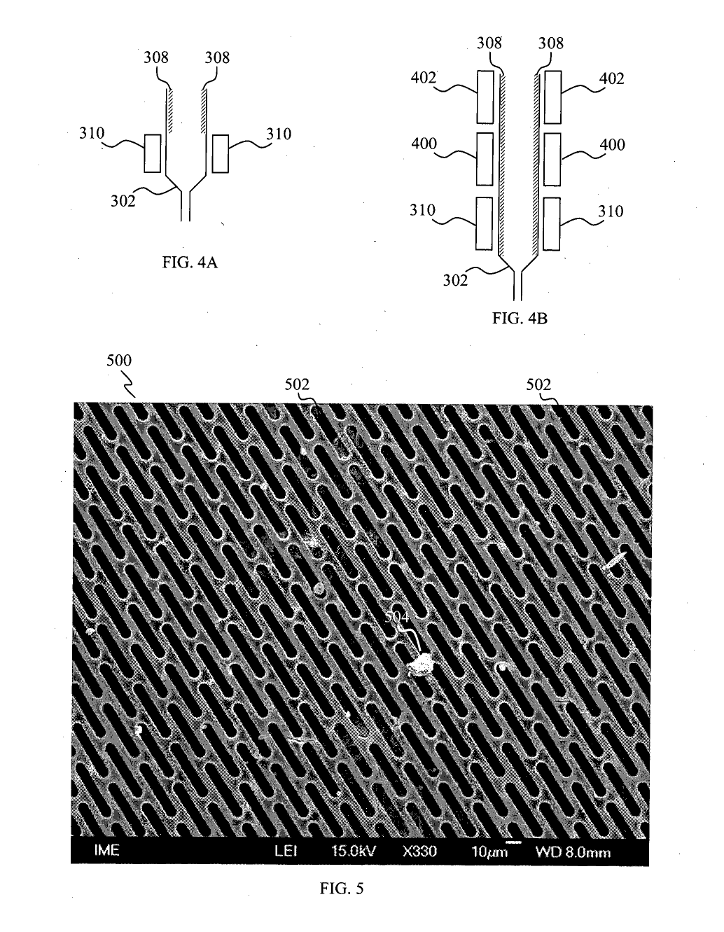

In the context of various embodiments, as shown in FIG. 4A, the layer 308 including leukocyte specific biomarkers may be coated on a section of the inner wall of the input chamber 302, and the magnetic element 310 may be arranged adjacent a portion of the input chamber 302 where the inner wall of the input chamber 302 is not coated with the layer 308. The magnetic element 310 may be movable and/or, activated and deactivated (e.g. using a electromagnet) as desired.

In the context of various embodiments, as shown in FIG. 4B, a plurality of magnetic elements 310, 400, 402, may be arranged along a length of the input chamber 302. In various embodiments, any one or each of the magnetic elements 310, 400, 402 may be movable and/or may be an electromagnet, which may be switched on (activated) and off (deactivated) as desired.

In the context of various embodiments, the layer 308 may include an azide, for example 4-azidoniline hydrochloride. The azide may also be an amino azide or aldehydic azide or epoxy azide or aromatic-fluoro-nitro azide.

In the context of various embodiments, the input chambers 302, 342 may have a length of between about 10 mm and about 200 mm, for example between about 10 mm and about 100 mm, between about 10 mm and about 50 mm or between about 100 mm and about 200 mm. The input chambers 302, 342 may have a width or a diameter of between about 10 mm and about 100 mm, for example between about 10 mm and about 50 mm or between about 50 mm and about 100 mm.

In the context of various embodiments, each of the input chambers 302, 342 may be a syringe or a vacutainer.

In the context of various embodiments, each of the filters 312, 332 may be configured to pass red blood cells, platelets and some leukocytes.

In various embodiments, a plurality of filters may be provided in the apparatus of various embodiments. For example, there may be consecutive filters arranged in or along the fluidic path such as to further differentiate between cells. For example, differentiating between nucleated and non-nucleated red blood cells, useful in isolation of fetal cells in maternal blood for non-invasive prenatal diagnostics. The plurality of filters may be provided together sequentially at a position of the fluidic path or may be spaced apart along the fluidic path.

In the context of various embodiments, each of the leukocyte specific biomarkers may include anti-CD45 specific antibodies.

In one embodiment, the filter, e.g. 312, 332, alone may be used in blood transfusion to remove tumor cells from circulation and purified blood returned back into body circulation as a means of therapy.

In the context of various embodiments, the biological entity includes or is a circulating tumour cell or a fetal cell or a stem cell. For example, the apparatus of various embodiments may be used to separate circulating tumour cells (CTCs) from the sample volume, by depleting leukocytes or white blood cells (WBCs), for example by means of the input chamber 302 based on immuno-affinity and/or immuno-magnetic separation, and depleting the red blood cells (RBCs) by means of the filter 312, 332.

The filters (e.g. microfabricated filter) 312, 332 may be a one-dimensional, two-dimensional or three-dimensional filter with ordered polygonal shapes or structures. The filter may be microfabricated, for example using lithography.

The filters (e.g. microfabricated filter) 312, 332 may be a single layer filter (e.g. FIG. 5). The filters 312, 332 may include a single porous layer including a plurality of pores, where each pore may have a diameter or dimension of between about 0.5 μm and about 30 μm, for example between about 0.5 μm and about 20 μm, between about 0.5 μm and about 10 μm or between about 5 μm and about 30 μm.

FIG. 5 shows a scanning electron microscope (SEM) image of a filter 500 having a porous layer including a plurality of pores 502. Each pore 502 may be an elongate pore or slit. Each pore 502 may have a dimension or a width of about 6 μm and a length of about 40 μm. As illustrated in FIG. 5, a biological entity (e.g. a tumour cell) 504 is captured by the filter 500.

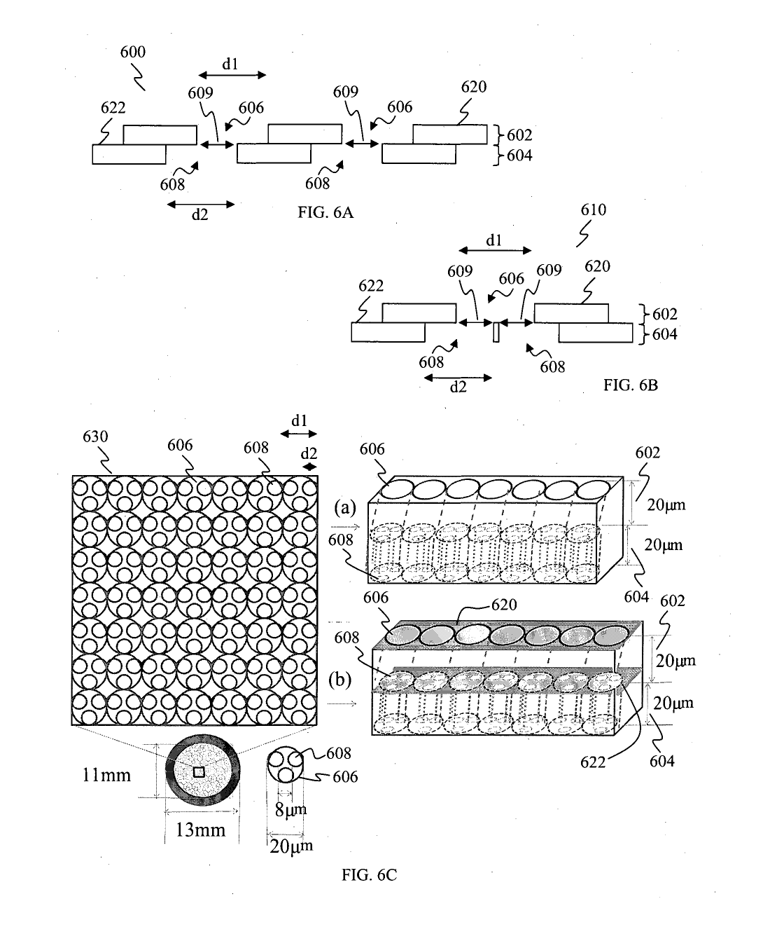

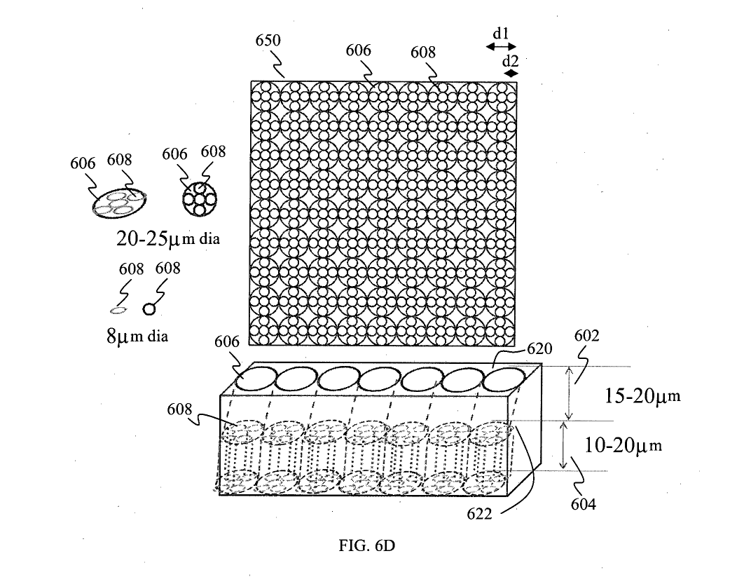

In various embodiments, the filters (e.g. microfabricated filter) 312, 332 may include two layers. FIGS. 6A and 6B show schematics of cross-sectional views of the filters 312, 332, according to various embodiments. The filters 312, 332 may have the configuration of the filter 600 of FIG. 6A or the filter 610 of the FIG. 6B.

Each of the filters 600, 610 may include a first porous layer 602 and a second porous layer 604 arranged one over the other, wherein the first porous layer 602 may include a plurality of first pores 606 defined through the first porous layer 602, wherein the second porous layer 604 may include a plurality of second pores 608 defined through the second porous layer 604, wherein one or more respective second pores 608 may be arranged to at least substantially overlap with each respective first pore 606 such that a respective opening 609 defined between a perimeter of the each respective first pore 606 and a perimeter of each of the one or more respective second pores 608 may be smaller than a diameter (or dimension), d1, of each first pore 606.

As illustrated in FIG. 6A, one respective second pore 608 may be arranged to at least substantially overlap with each respective first pore 606 such that a respective opening 609 defined between a perimeter of the each respective first pore 606 and a perimeter of the one respective second pore 608 may be smaller than a diameter (or dimension), d1, of each first pore 606.

As illustrated in FIG. 6B, a plurality of respective second pores 608 may be arranged to at least substantially overlap with each respective first pore 606 such that a respective opening 609 defined between a perimeter of the each respective first pore 606 and a perimeter of each of the plurality of respective second pores 608 may be smaller than a diameter (or dimension), d1, of each first pore 606.

In various embodiments, each second pore 608 may have a diameter (or dimension), d2, that is equal to or smaller than the diameter (or dimension), d1, of each first pore 606.

In embodiments where each second pore 608 has a diameter (or dimension), d2, that is smaller than the diameter (or dimension), d1, of each first pore 606, the one or more respective second pores 608 may be arranged to be within the perimeter of each respective first pore 606.

In the context of various embodiments, d1 may be between about 5 μm and about 30 μm, e.g. between about 10 μm and about 20 μm or between about 20 μm and about 25 μm, while d2 may be between about 0.5 μm and about 15 μm, e.g. between about 5 μm and about 15 μm, between about 0.5 μm and about 5 μm or between about 0.5 μm and about 10 μm.

FIGS. 6C and 6D show schematics of the filters 312, 332, according to various embodiments. The filters 312, 332 may have the configuration of the filter 630 of FIG. 6C or the filter 650 of the FIG. 6D.

Each of the filters 630, 650 may include a first porous layer 602 and a second porous layer 604 arranged one over the other, wherein the first porous layer 602 may include a plurality of first pores 606 defined through the first porous layer 602, wherein the second porous layer 604 may include a plurality of second pores 608 defined through the second porous layer 604, and wherein a plurality of respective second pores 608 may be arranged to at least substantially overlap each respective first pore 606 such that a respective opening defined between a perimeter of the each respective first pore 606 and a perimeter of each of the plurality of respective second pores 608 may be smaller than a diameter, d1, of each first pore 606. As shown in FIGS. 6C and 6D, the plurality of respective second pores 608 may be arranged to be within a perimeter of each respective first pore 606.

In the context of various embodiments, the first porous layer 602 may be in contact with the second porous layer 604. In further embodiments, the first porous layer 602 may be spaced apart from the second porous layer 604 by a gap.

In the context of various embodiments, the first porous layer 602 may have a thickness of between about 1 μm and about 20 μm, e.g. between about 1 μm and about 10 μm, between about 1 μM and about 5 μm or between about 10 μm and about 20 μm. The second porous layer 604 may have a thickness of between about 1 μm and about 20 μm, e.g. between about 1 μm and about 10 μm, between about 1 μm and about 5 μm or between about 10 μm and about 20 μm.

In the context of various embodiments, each first pore 606 may have a diameter between about 5 μm and about 30 μm, e.g. between about 10 μm and about 20 μm or between about 20 μm and about 25 μm. Each second pore 608 may have a diameter between about 0.5 μm and about 15 μm, e.g. between about 5 μm and about 15 μm, between about 0.5 μm and about 5 μm or between about 0.5 μm and about 10 μm.

In the context of various embodiments, the plurality of first pores 606 may be uniformly distributed on the first porous layer 602. The plurality of second pores 608 may be uniformly distributed on the second porous layer 604.

In the context of various embodiments, three second pores 608 may be arranged to at least substantially overlap with each first pore 606. The three second pores 608 may be arranged in a form resembling ‘Y’.

In various embodiments, the three second pores 608 may be completely arranged within the perimeter of each first pore 606 beneath the each first pore 606. FIG. 6C shows a non-limiting example.

In the context of various embodiments, five second pores 608 may be arranged to at least substantially overlap with each first pore 606. The five second pores 608 may be arranged in a form resembling ‘X’.

In various embodiments, the five second pores 608 may be completely arranged within the perimeter of each first pore 606 beneath the each first pore 606. FIG. 6D shows a non-limiting example.

In the context of various embodiments, the first porous layer 602 has a top surface 620 and a bottom surface, and wherein the second porous layer 604 has a top surface 622 and a bottom surface, the top surface 622 of the second porous layer 604 facing the bottom surface of the first porous layer 602, and wherein the top surface 620 of the first porous layer 602 includes a metal layer. In various embodiments, the top surface 622 of the second porous layer 604 includes a metal layer. Each metal layer of the top surface 620 of the first porous layer 602 and the top surface 622 of the second porous layer 604 includes a metal selected from the group of gold, silver or copper.

In the context of various embodiments, each first pore 606 may have a shape selected from the group consisting of a circle, an oval, a hexagon, a square and a rectangle. Each second pore 608 may have a shape selected from the group consisting of a circle, an oval, a hexagon, a square and a rectangle.

In the context of various embodiments, at least one of the first porous layer 602 or the second porous layer 604 may include or may be made of parylene or silicon dioxide or silicon nitride or silicon or other materials used in microfabrication.

In further embodiments, the filters 312, 332 may include a plurality of first channels arranged in a first row, and a plurality of second channels arranged in a second row adjacent to the first row, wherein one or more respective second channels may be arranged to at least substantially overlap with each respective first channel such that a respective opening defined between an edge of the each respective first channel and an edge of each of the one or more respective second channels is smaller than a width of each first channel. In further embodiments, the filters 312, 332 may further include a plurality of third channels arranged in a third row, wherein the second row may be arranged between the first row and the third row, and wherein one or more respective third channels may be arranged to at least substantially overlap with each respective second channel such that a respective opening defined between an edge of the each respective second channel and an edge of each of the one or more respective third channels may be smaller than a width of each second channel.

Non-limiting examples of the apparatus and method for separating a biological entity (e.g. CTCs) from a sample volume, with related sample preparation, fabrication method and related measurements will now be described.

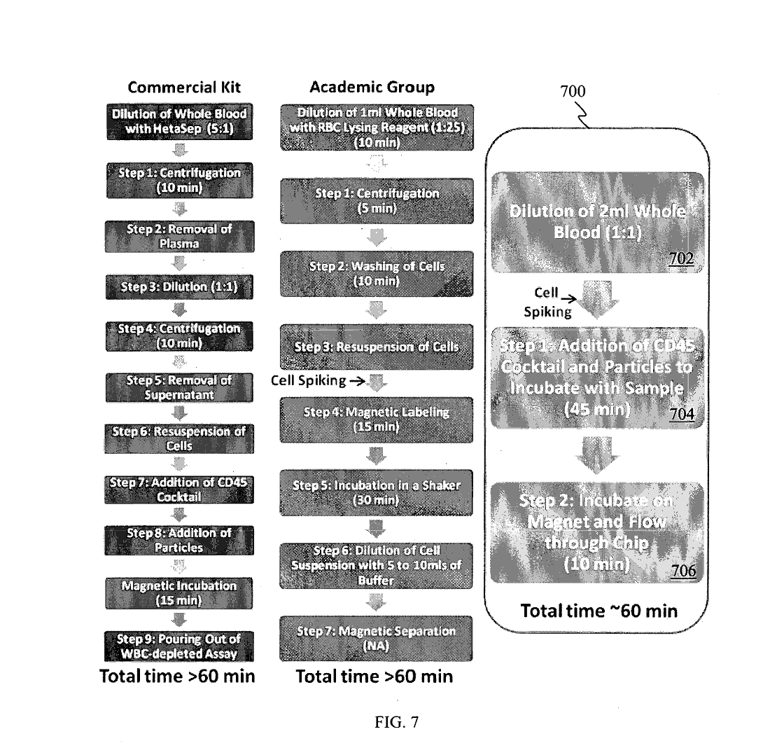

Negative enrichment may provide a suitable approach for tumor cell isolation as it does not rely on biomarker expression. However, size-based negative enrichment methods suffer from well-known recovery/purity trade-off. Non-size based methods have a number of processing steps that may lead to compounded cell loss due to extensive sample processing and handling which result in a low recovery efficiency. Thus, there is a need for the development of reliable, efficient platform to isolate, enrich and characterize CTCs in blood. In view of this, various embodiments may provide a method that may perform negative enrichment in 2 steps. As non-limiting examples, negative enrichment may be performed from 2 ml of whole blood in a total assay processing time of 60 minutes without using centrifugation or chemical means, as generally shown in FIG. 7, and as compared to conventional methods of negative selection for CTC isolation process.

For the method 700 of various embodiments, at 702, approximately 2 ml of whole blood obtained may be diluted, for example with a cellular preservative, at a ratio of (1:1).

At 704, a mixture of CD45 and magnetic particles may be added to obtain a sample. Cell spiking may be carried out prior to the addition of the mixture.

At 706, the sample may be incubated on magnet and flowed through a chip (e.g. microfluific chip) to separate the CTCs from other blood constitutents.

The method 700 may be performed in a total time of approximately 60 minutes.

As shown in FIG. 7, as compared to conventional methods, for example as performed using a commercial kit based on a 9-step approach or performed by the academic group based on a 7-step approach, the method 700 requires a reduced number of steps and a reduced processing time, thereby reducing the multiple handling processes to two steps to reduce the risks of losing target cells. The method 700 may simplify assay implementation with elimination of several steps, while offering better performance.

It should be noted that while cell spiking happens mid-way through the protocol indicated for the “Academic Group”, this is not fully representative of the comprehensive cell loss of the entire process flow.

### Example 1

Various embodiments may provide a microfluidic platform for negative enrichment of circulating tumor cells (CTCs). Various embodiments may employ an approach based on the method 700 of FIG. 7.

The microfluidic negative selection platform may combine a single-step WBC and chemical-free RBC depletion approach without manual sample transfer. The immunomagnetic WBC depletion may be employed directly in approximately 2 mL of whole blood. The WBC-depleted blood may flow through a microfluidic chip that contains a precision-manufactured micro slit membrane. The micro slit membrane may be designed to selectively allow passage of RBCs while retaining as many nucleated cells. Thus, this method may deplete WBCs and RBCs in a single step without the use of centrifugation or chemical lysis. In addition, it may avoid or minimise multiple sample handling. Combining the negative depletion of WBCs with blood filtration, as will be discussed below, more than 90% WBC depletion and greater than 90% recovery of CTCs may be achieved in a fast turnaround time of an hour. As will be described later, spiking experiments show an average of >90% recovery of cancer cells over a range of spiked cell numbers for multiple cell lines. In addition, since the sample is not subjected to any chemical manipulation, the exit volume may be used for complimentary assays to extract molecular information such as serum protein or nucleic acid assays. Circulating tumour cells (CTCs) have also been successfully recovered from approximately 2 ml of clinical cancer patient samples.

Sample Preparation

Blood was drawn from healthy donors of both genders into 6-mL 25 BD Vacutainer K2 EDTA tubes (Becton Dickinson). Samples drawn had a cellular preservative (Catlog#213358, Streck) manually added immediately after blood draw at a ratio of 1:1 and were maintained at about 4° C. The healthy donors had no known illness or fever at the time of draw and no history of malignant disease. Cancer patients' samples were obtained from National University Hospital (Singapore) under Institutional Review Board (IRB) approval. Similarly, the blood was drawn into 6-mL BD Vacutainer K2 EDTA tubes followed by the addition of cellular preservative as described above. Samples drawn were maintained in a 4° C. environment from the point of collection to the place of processing and were processed within 48 hours from the time of draw.

At the beginning of each experiment, approximately 4 ml of preservative-added blood assay (1:1::blood:preservative) was pipetted from the vacutainer tube into a 5-mL BD syringe barrel (Catlog#302135, 40 USA). A mixture of 100 μl of customized anti-CD45 Tetrameric Antibody Complexes (TAC) (StemCell Technologies, Canada) and 100 μl of customized magnetic particles (StemCell Technologies Inc., Canada) was added to the assay and incubated for 30 min. Subsequently, “The Big Easy” EasySep® Magnet was placed around the barrel to separate the labelled WBCs. Subsequently, the WBC-depleted assay was put through a micro slit membrane for target cell isolation. WBC depletion was determined by counting the cells before and after the immunomagnetic depletion procedure using an automated clinical haematology analyzer Horiba Micro ES60. (Horiba, Japan).

Microslit Membrane

A micro slit membrane was designed to deplete platelets, RBCs, smaller-sized WBCs, such as lymphocytes, monocytes, and granulocytes that escaped immunomagnetic depletion, while retaining the majority of the other nucleated cells. A 10-μm thick micro slit membrane was fabricated from Parylene-C. The membrane was circular in shape with a diameter of approximately 13-mm and an active diameter of approximately 9-mm. The membrane includes periodically arranged precise slits with a slit dimension of approximately 5.5-μm in width and 40-μm in length. This design was chosen after comparing it with other micro filter designs for cell separation due to its advantages in retaining CTCs. Compared to commonly used circular/hexagonal microfilters openings, the rectangular slit design has the advantage of defraying the pressure applied across the cells trapped on the membrane and preserving the viability and morphology of target cells. In addition, this micro slit membrane configuration has a large fill factor (39%), which combined with upstream WBC depletion, may allow blood flow at very small air pressure of approximately 3.5 mbar.

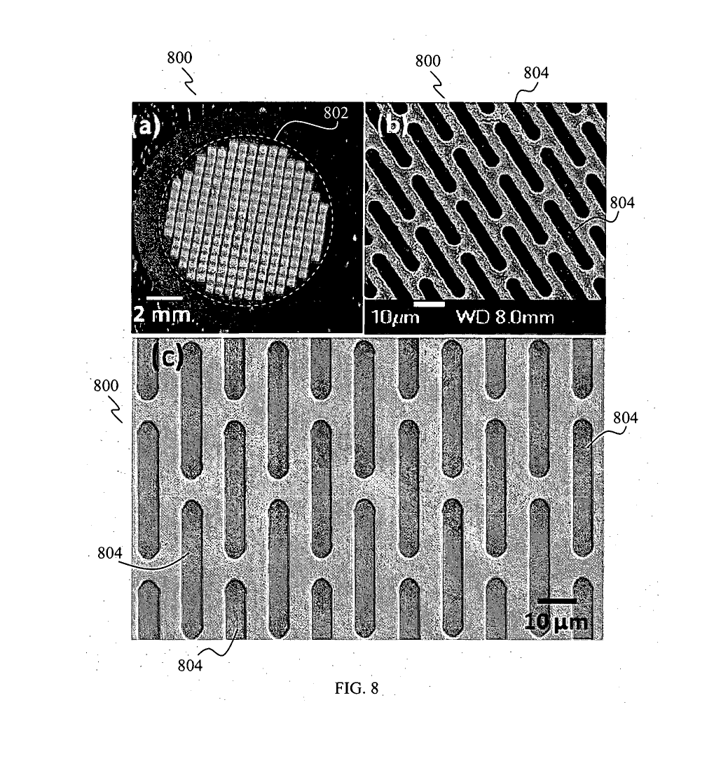

FIG. 8 illustrates the different views of the Parylene micro slit membrane 800 of approximately diameter 13 mm, with an active diameter as represented within the dotted circle 802, and containing slits or elongate pores, as represented by 804 for some slits, with dimension approximately 5.5×40 μm and a fill factor of approximately 39%. FIG. 8(a) shows an overview picture of the membrane 800, FIG. 8(b) shows an image taken at 300X using scanning electron microscope (SEM), and FIG. 8(c) shows an image taken at 50X using optical microscope.

Microfluidic Chip

A precision-manufactured circular Parylene-C micro slit membrane was packaged in a microfluidic chip. Polymethyl methacrylate (PMMA) was used for substrate material because of its characteristics of good light transition and high chemical resistance. The chip is approximately 25-mm wide and approximately 75-mm long, which is the same size as a regular microscope slide, and, it can be easily mounted on the stage of microscope for inspection.

A polymer mesh was integrated underneath the membrane as a support to prevent or minimise any potential deformation while the Parylene-C micro slit membrane (e.g. 800, FIG. 8) was subjected to positive pressure. The mesh grid size was approximately 250 μm×250 μm, which did not cause any clogging of cells.

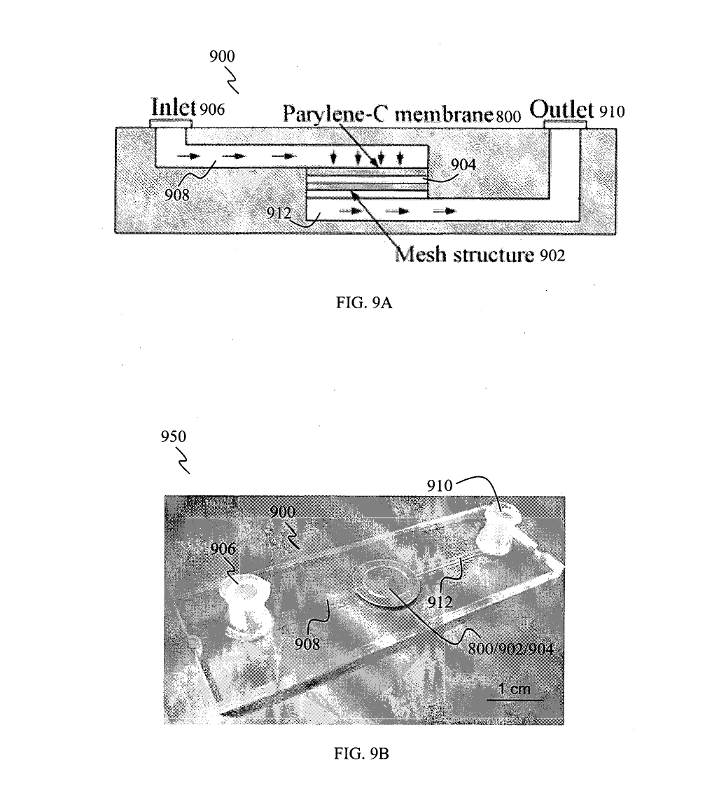

FIG. 9A shows as cross sectional view of a microfluidic chip 900, while FIG. 9B shows a photograph image 950 of the microfluidic chip 900 of the embodiment of FIG. 9A, according to various embodiments. The microfluidic chip 900 includes the Parylene-C micro slit membrane 800 and a polymer mesh 902 arranged below the membrane 800, where the membrane 800 and the polymer mesh 902 may be arranged in a microfluidic chamber 904. The microfluidic chip 900 includes an inlet 906 which may be coupled to and in fluid communication with a microchannel 908, which in turn is in fluid communication with the microfluidic chamber 904. The microfluidic chip 900 further includes an outlet 910 which may be coupled to and in fluid communication with a microchannel 912, which in turn is in fluid communication with the microfluidic chamber 904. A sample may flow in the direction from the inlet 906 towards the microfluidic chamber 904 where the membrane 800 may be positioned therewithin, and towards the outlet 910, as represented by the arrows.

During processing, the inlet 906 of the chip 900 was connected to a 5-mL syringe barrel (not shown) for sample assay introduction. A 4-mL sample assay was delivered by the connecting microchannel 908, which may be approximately 1-mm wide, to the chamber 904 with the micro slit membrane 800 for target cell isolation. Subsequently, the waste from the outlet 910 of the chip 900 was collected in a 15-mL Falcon tube (Catlog#352097, BD). The microfluidic chip 900 provided an integrated and enclosed platform for sample manipulation, which avoided the risks of cell loss associated with sample transferring and handling.

Prior to the experiment, each chip, e.g. 900, was microscopically inspected for any defects. The channels e.g. 908, 912 and the membrane chamber e.g. 904 were primed with 1×PBS (Catlog#10010, Invitrogen) to remove any air bubbles before the blood sample was introduced into the chip.

Cell Culture

Human lung cancer cell line NCI-H 1975 and human breast cancer cell line MCF-7 were purchased from American Type Culture Collection (Manassas, Va.). NCI-H1975 cells were cultured in RPMI 1640 (Catlog#22400-089, Invitrogen) supplemented with 10% Fetal Bovine Serum (FBS) (Catlog#04-001-1A, Biological Industries). MCF-7 cells were cultured in DMEM (Catlog#11965-092, Invitrogen) supplemented with 10% FBS. Cells were maintained at 37° C. in a humidified atmosphere containing 5% CO₂ and harvested with trypsin before use. The cell suspensions were used only when their viability as assessed by trypan blue exclusion exceeded 90%.

Cell Spiking

To demonstrate the sensitivity and linearity of the rare cell isolation assay, two sets of four 2-mL aliquots of peripheral blood diluted with 2 mL of cellular preservative were prepared. The first set (Set A) of four aliquots was spiked with a titration series of NCI-H1975 cells with approximately 10, 30, 50, or 100 cells per 2-mL blood. The second set (Set B) of four aliquots was spiked with a similar titration series as the first but with MCF-7 cells. For the reproducibility assay, two additional sets of replicates were prepared for Sets A and B. Spiking of cancer cells into healthy donor blood was conducted before immunomagnetic WBC depletion step to fully mimic the process flow with patient blood.

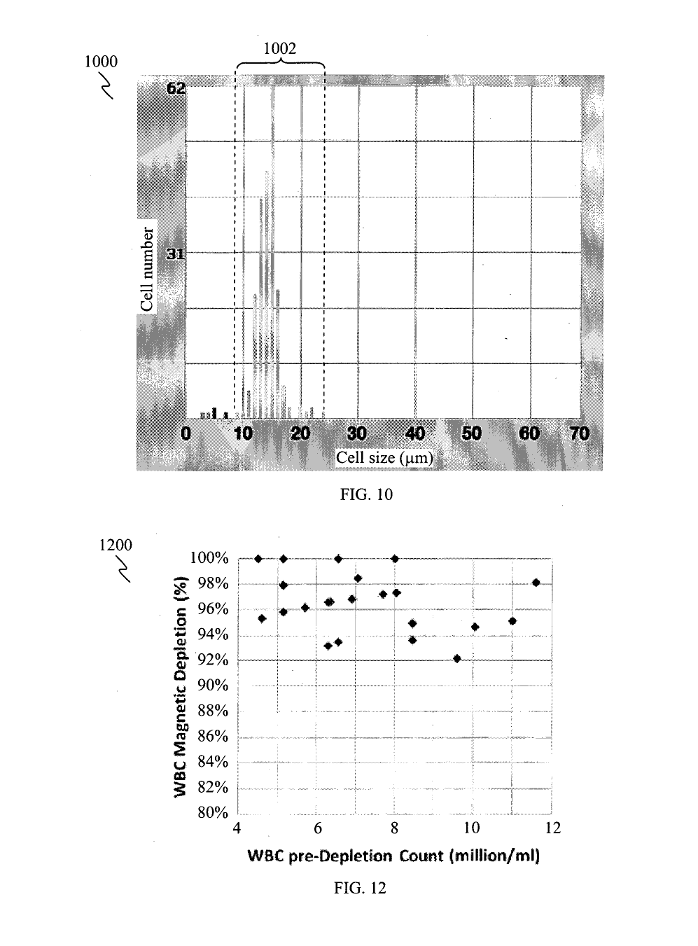

FIG. 10 shows a plot 1000 of size distribution of cultured NCI-H1975 lung cancer cell line, according to various embodiments, where data was obtained using an automated cell counter (Luna, LogosBiosystems). It may be observed that the sizes of cells spiked were ranging from approximately 9 μm to 25 μm, represented by 1002. It is important to ensure that a good CTC isolation system may be capable of capturing cells with a wide range of cell sizes in order to achieve high isolation efficiency. Cells were not fixed prior to isolation.

Staining Protocol

To identify NCI-H1975 and MCF-7 cells on the precision micro-slit membranes 800, a mouse anti-pan Cytokeratin monoclonal antibody conjugated with fluorescein isothiocyanate (FITC) (Catlog#ab78478, Abeam) for cancer cells and a mouse anti-Human CD45 monoclonal antibody conjugated with Phycoerythrin(PE)-Dyomics 590 (Catlog#CLX48PE-DY590, Cedarlane Laboratories) for hematologic cells were used. After the microfiltration process, approximately 500 μL of 4% paraformaldehyde (PFA) (Catlog#P6148, Sigma) in 1×PBS was introduced to the chip 900 and incubated for about 15 minutes to fix the samples on the micro slit membrane 800. Approximately 1 mL of 1×PBS was then introduced to rehydrate the samples. The samples were then permeabilized with 0.25% Triton X-100 (Catlog#X100, Sigma) in 1X PBS and incubated for about 10 minutes. The samples were then washed with approximately 1 mL of 1×PBS. After blocking non-specific binding sites with 5% bovine serum albumin (BSA) (Catlog#A2153, Sigma) in 1×PBS for about 30 minutes, the chip 900 was incubated for about 30 minutes with anti-pan Cytokeratin (FITC) and anti-Human CD45 (PE-Dyomics 590) antibodies in a dark room. The samples were then washed with approximately 1 mL of 1×PBS. After counter-staining with approximately 1 μL of Hoechst 33342 (Catlog#H1399, Invitrogen) in approximately 499 μL of 1×PBS, the samples were washed for the last time with approximately 1 mL of 1×PBS to minimize background noise. Finally, the chip 900 was mounted on an upright fluorescent microscope (BX61, Olympus) for CTC enumeration and sample analysis.

Experimental Setup

FIG. 11A shows a schematic perspective view of an apparatus 1100 for CTCs isolation from whole blood, illustrating the components in the CTC isolation system and microfluidic chip setup 1100. FIG. 11B shows a photograph image of an entire set-up or system 1140 for CTC isolation, including the apparatus 1100, and additional components such as a pump 1150, and a cassette 1152 for securing the chip 900. The sample may be driven by a pneumatic pressure regulated precisely by the pump 1150.

The setup 1100 includes a functionalised syringe barrel, e.g. a 5-ml syringe barrel 1102 containing blood sample conjugated with TAC and magnetic particle complex, placed within a magnet 1104 for immunomagnetic WBC depletion. The syringe barrel 1102 with the surrounding magnet 1104 were placed directly above another 5-mL syringe barrel 1106, for collecting WBC depleted sample. A valve 1108 may be arranged in between the syringe barrel 1102 and the syringe barrel 1106. The syringe barrel 1106 was connected to the inlet 906 of the microfluidic chip 900. WBCs within the sample were depleted by immunomagnetic separation before being flowed down to the chip 900 to retain CTCs. This means that upon completion of the magnetic incubation period, the WBC-depleted blood was flowed through the chip setup 900 under gravity. A tubing or conduit 1110 may be connected to the outlet 910 of the chip 900 for transfer of the sample to waste after passing thorough the chip 900.

Subsequently, the WBC-depleted assay was driven by a constant 3.5-mBar air pressure (e.g. pump 1150; MV20 pump, Ibidi) through the microfluidic chip 900 for microfiltration process. The applied pressure by the pump 1150 was monitored in real time by pump controller software.

The microfiltration process of 4-ml blood assay was completed in less than 5 minutes. Approximately 2 mL of 1λ PBS was introduced and flowed continuously to wash and remove the remaining RBCs on the micro slit membrane 800 in the microfluidic chamber 904. Fixation of cells followed by the staining protocol was initiated after the washing step. The chip 900 was then removed from the connectors and inspected under a fluorescent microscope. The same 3.5 mBar air pressure was employed to deliver the reagents in all of the procedures, including washing, fixation, and staining steps.

Cell Identification

The inspection process was conducted by fluorescence microscopy using an upright microscope (BX61, Olympus) with a motorized xy stage. Image capturing was performed by 14-bit monochrome ExiAqua fast 1394 CCD camera (Qlmaging, Canada). U-MWU2, U-MWIB2, and U-MSWG2 filter sets were used to visualize staining of Hoechst, FITC, and PE-Dyomics 590 probes. Motorized stage was controlled by a joystick to scan through the entire micro slit membrane. In addition, the software, Image Pro-Plus MDA (Media Cybernetics, USA), was employed to apply pseudo color to the acquired cell images. Cell counting and image capturing were performed using a 40× objective lens. The cell identification process was performed by an experienced molecular biologist specialized in cell pathology.

A CTC was defined as an object with the following criteria: (a) circular to oval morphology, (b) a visible nucleus (Hoechst positive), (c) negative staining for CD45, and (d) positive staining for Cytokeratin. Results of cell enumeration are always expressed as the number of cells per 2-mL of blood.

Cell Depletion and Recovery

The depletion of WBCs by immunomagnetic method, cancer cell recovery and total WBC depletion on the micro slit membrane were determined as follow:

$$\begin{matrix}WBC depletion ( \% ) = \frac{{W}_{i} - {W}_{f}}{{W}_{i}} \times 100 , & ( Equation 1 ) \\ Recovery efficiency ( \% ) = \frac{{C}_{R}}{{C}_{S}} \times 100 , & ( Equation 2 ) \\ Total WBC depletion ( log ) = log ( \frac{{N}_{i}}{{N}_{f}} ) . & ( Equation 3 )\end{matrix}$$

Wᵢ is the total number of WBC in the original 2-mL sample and W_{f} is the number of WBC in sample after immunomagnetic WBC depletion. Both Wᵢ and W_{f} were determined by an automated clinical haematology analyzer. Cₛ is the number of cancer cells spiked into the 4-mL blood assay prior to immunomagnetic WBC depletion process when C_{R} is the number of cancer cells isolated and counted on the micro slit membrane through fluorescent microscopy. Nᵢ is the total number of nucleated cells in the sample prior to the experiment, which is equal to Wᵢ, and, N_{f} is the number of nucleated cells counted on the micro slit membrane by a Matlab-based automated image-processing algorithm, CellC.

Results and Discussions

Upstream Immunomagnetic WBC Depletion

A simple yet effective upstream immunomagnetic WBC depletion method, directly in whole blood, has been developed. The results of WBC depletion efficiency using the modified protocol of various embodiments are shown in FIG. 12.

The numbers of WBCs in these experiments ranged between about 4.5 million/mL and about 11.6 million/mL. By employing the developed methodology, an average of 97% of WBCs was depleted before reaching the micro slit membrane using only approximately 50 μL of antibody per mL of blood. Due to a high degree of WBC depletion before microfiltration, combined with a larger membrane area and a high fill factor, the blood filtration process was accomplished in less than five minutes at a minimal positive pressure of 3.5-mbar. The process resulted in excellent morphology of retained cells. The total WBC depletion, from negative selection to cell isolation at the membrane surface was determined to be 2.3 log.

On-Chip Cell Counting and Isolation Efficiency

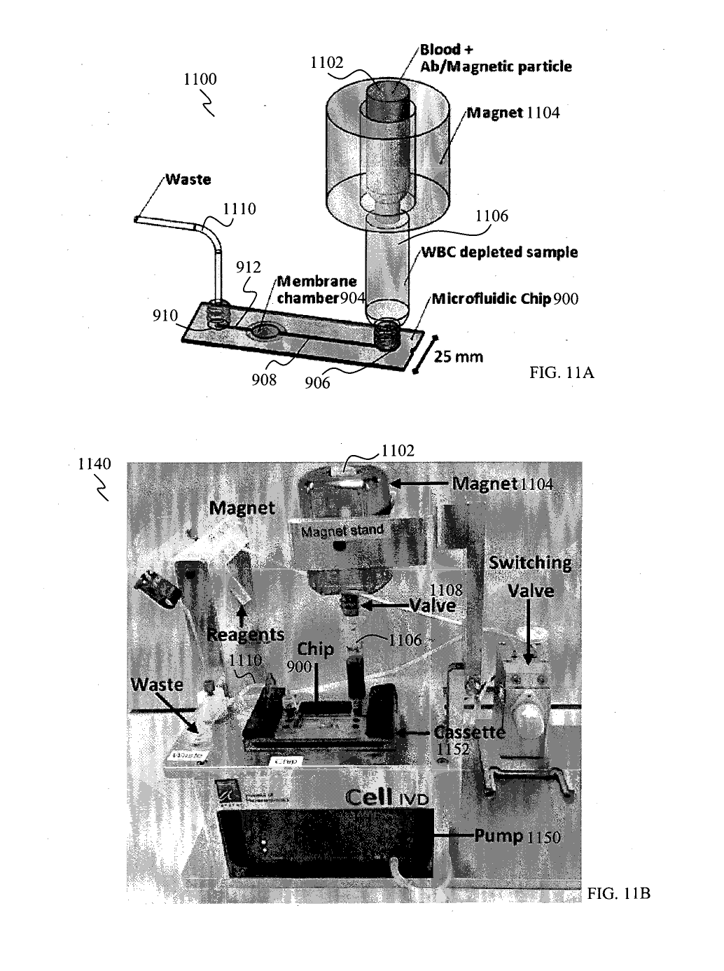

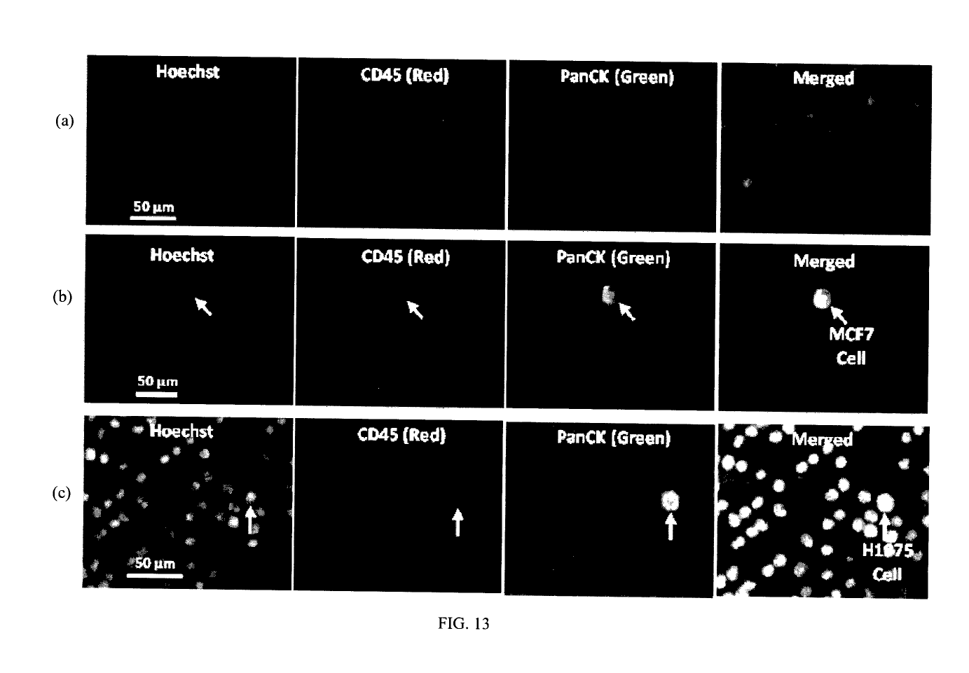

Negative controls were performed alongside experiments with spiked cancer cells. Results of isolation and enumeration of spiking cancer cells are shown in FIG. 13, where the nucleuses were stained with Hoechst (blue), the WBCs were stained by PE-labelled anti-CD45 (red) and the cancer cells were stained by FITC-labelled antibodies Pan-Cytokeratin (green).

FIG. 13(a) shows images taken under three different filters, for negative control, healthy donor blood without spiking any cancer cells. Cells that were Hoechst-positive, CD45-negative and panCK-positive were considered as a cancer cell. No cancer cells were found in healthy donor blood samples.

Next, various concentrations of cell lines were spiked in 2-mL of whole blood and processed through the cell isolation system 1140 (FIG. 11B). The isolated cancer cells and other nucleated cells were retained on the micro slit membrane 800 after the microfiltration process. The isolated target cells were fixed and stained according to the above described staining protocol for CTC identification and counting.

FIG. 13(b) shows images taken under three different filters, for healthy donor blood that was spiked with MCF7 cells, while FIG. 13(c) shows images taken under three different filters, for healthy donor blood that was spiked with NCI-H1975 cells, to indicate the cancer cell as Hoechst-positive, CD45-negative and pan-CK-positive. In addition, the merged images of the respective images corresponding to Hoechst, CD45 and panCK are also provided in FIG. 13.

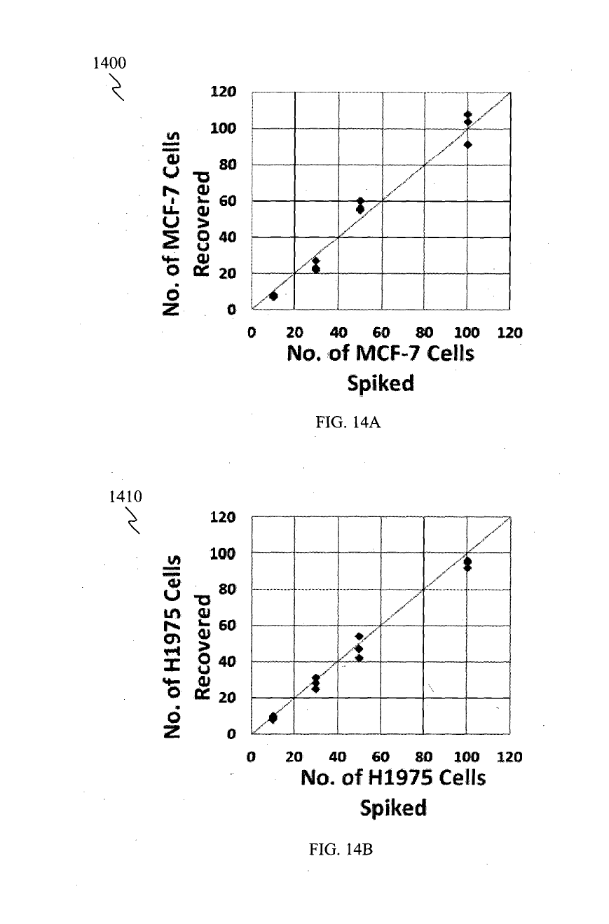

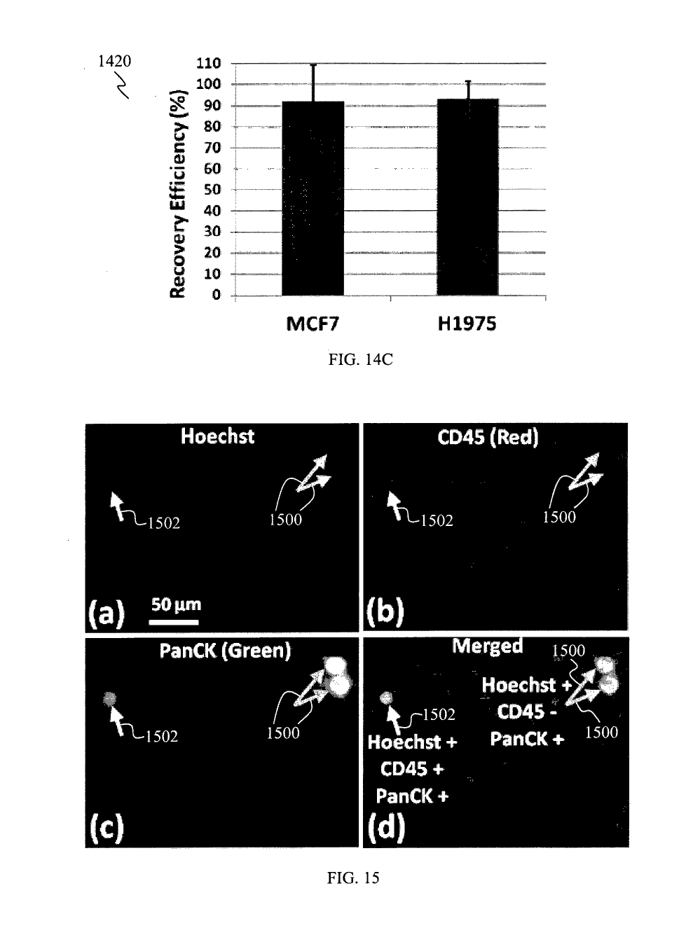

FIGS. 14A and 14B show plots 1400, 1410 showing respectively the number of MCF-7 cancer cells and NCI-H1975 cancer cells, recovered and counted on the micro slit membrane as a function of number of cancer cells spiked into the 4-mL sample assay. FIGS. 14A and 14B show the results for the cells recovered from whole blood as a function of four different spiking concentrations (n=3).

An average of 92.17% of MCF-7 were recovered across the spiking range of 10 to 100 cells (n=3 for each concentration), and an average of 93.25% of NCI-H1975 was recovered from using the system 1140 in the same spiking range of 10 to 100 cells, as shown in the plot 1420 of FIG. 14C.

Through these experiments, non-specific cells that were positive for all three stains were observed. FIG. 15 shows fluorescence images under different filters for cells identification and classification, for Hoechst stains cell nucleus (FIG. 15(a)), PE-labelled anti-CD45 antibody stains CD45+ cells (WBCs) (FIG. 15(b)), FITC-labelled PanCK antibody stains PanCK+ cells (cancer cells) (FIG. 15(c)) and a merged image (FIG. 15(d)) based on the images of FIGS. 15(a), 15(b) and 15(c). The arrows 1500 indicate the cancer cells that are Hoechst-positive CD45-negative and PanCK-positive while the arrow 1502 indicates the unknown artefact cell that is Hoechst-positive CD45-positive and PanCK-positive. The rest of the cells in the FIG. 15(d) are WBCs, which are Hoechst-positive CD45-positive and PanCK-negative. As may be observed in FIG. 15, both non-specific cells and cancer cells were clearly distinguished.

On-Chip Purity

The numbers of WBCs were recorded before and after immunomagnetic depletion, as discussed above, using an automated clinical haematology analyzer. The final assay purity was determined by counting the number of nucleated cells retained on the micro slit membrane alongside cancer cells.

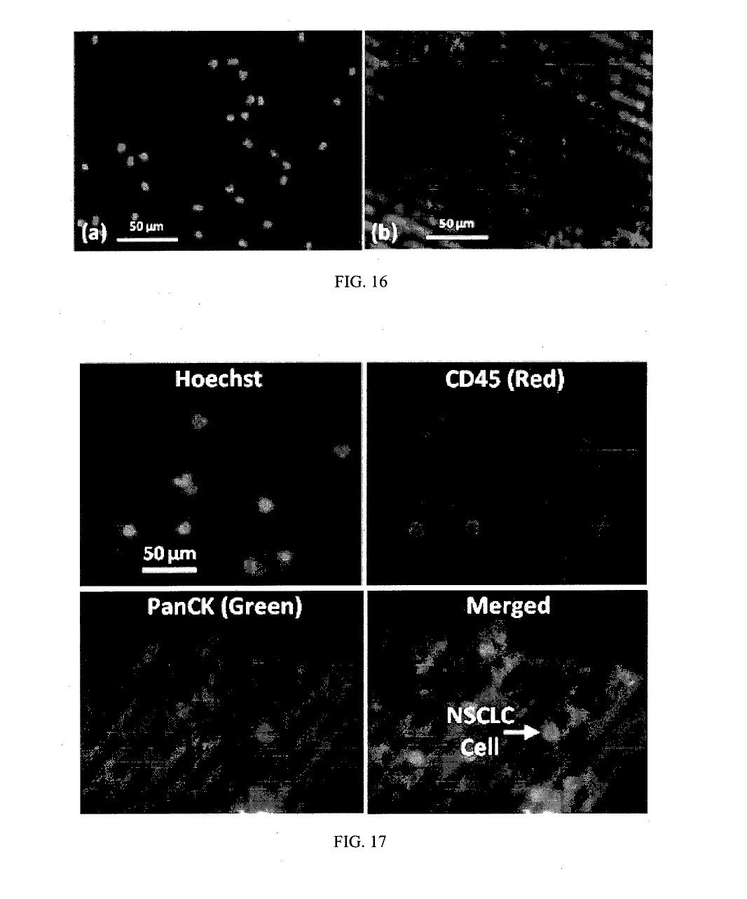

FIG. 16 shows Hoechst-stained fluorescent images of nucleated cells (nucleus of peripheral blood mononuclear cells (PBMCs)) captured by a micro slit membrane, where FIG. 16(a) shows the results for an experiment accomplished with immunomagnetic WBCs depletion (WBC-depleted blood sample) as described above, which shows only few nucleated cells on the membrane, while FIG. 16(b) shows the results for an experiment accomplished without immunomagnetic WBCs depletion, where layers of WBCs were captured on the membrane which lead to a lower purity.

The experiment accomplished without immunomagnetic WBCs depletion took more than 30 minutes to finish the microfiltration process on the microfluidic chip 900. The images clearly demonstrate that the WBC-depleted sample has a much lower number of nucleated cells on the micro slit membrane than one without depletion, which explains the smoother sample flow and higher purity of cancer cell enrichment by the system 1140. No RBCs were observed on the membrane surface. Automated imaging and image processing algorithms were employed to acquire high magnification images of Hoechst-stained cells to determine the total number of nucleated cells isolated on the micro slit membrane 800. Using this method, the total nucleated cell depletion was determined to be 2.3 log₁₀.

Clinical Testing

To demonstrate the utility of the apparatus 1100 or the system 1140 in detecting CTCs from clinical patient samples, five clinical samples were received from National University Hospital, Singapore, to be processed on the apparatus 1100 or the system 1140. These included four Non-Small-Cell Lung Carcinoma (NSCLC) and one colorectal (CRC) cancer case. These samples were processed through the CTC isolation and enumeration system 1140. The system 1140 successfully detected CTCs in 5 out of 5 patients based on the cell identification criteria of nucleus stain Hoechst-positive, CD45-negative and pan-CK-positive. Table 1 summarizes the results from the clinical testing.

TABLE 1
Cancer cell isolation and WBC depletion data by processing
2 mL of whole blood from 5 cancer patients

| | | | | Immuno- | | |
|---|---|---|---|---|---|---|
| | | Pre | Post | magnetic | # of | |
| | | Count | Count | WBC | CTCs | Total |
| | Cancer | WBC | WBC | depletion | (per | WBC |
| Patient | Type | (M/mL) | (M/mL) | (%) | 2 mL) | Depletion |
| 1 | NSCLC | 6.55 | 0.105 | 98.10 | 22 | 2.76 log |
| 2 | NSCLC | 8.01 | 0.080 | 99.01 | 15 | 2.79 log |
| 3 | NSCLC | 11.6 | 0.215 | 98.15 | 1 | 2.46 log |
| 4 | NSCLC | 11.0 | 0.538 | 95.11 | 12 | 2.61 log |
| 5 | CRC | 9.61 | 0.753 | 92.16 | 11 | 2.40 log |

FIG. 17 shows the fluorescent images (Hoechst-stained fluorescent image, PE-labelled anti-CD45-stained fluorescent image, FITC-labelled antibodies Pan-Cytokeratin-stained fluorescent image, and a merged image of the above-mentioned stained fluorescent images) imaged under three different filters for fluorescent microscopy, taken from one of NSCLC patient sample. As may be observed in FIG. 17, a CTC (NSCLC cell) from the NSCLC patient sample was captured on the micro slit membrane.

As described above, a simple, 2-step negative enrichment protocol for CTC isolation has been demonstrated. This method does not rely on either antigen expression, nor employs centrifugation and other extensive sample handling steps, which otherwise may lead to compounded cell loss and may be difficult to standardize. In the approach of various embodiments, an effective upstream immunomagnetic WBC depletion method has been developed to deplete WBCs directly in whole blood. This may be coupled with a downstream precision micro-slit membrane that may perform a chemical-free and centrifugation-free RBC depletion as well as highly efficient CTC retention. In various embodiments, it should be appreciated that a layer including leukocyte specific biomarkers may be coated on at least a section of an inner wall of the syringe barrel 1102, where the leukocyte specific biomarkers may also couple or bind to leukocytes (WBCs) present in the blood sample.

The technique as described yields one or more benefits such as (a) highly efficient, unbiased isolation of unfixed cells with excellent morphology, (b) fast turnaround time, (c) scalable, (d) amenable to full automation (hence standardization), and (e) 2.3 logᵢₐ total WBC depletion which may enable routine downstream molecular analysis. Besides demonstrating high recovery from low spiked cell numbers, up to 100% CTC detection in clinical patient samples has been demonstrated.

The apparatus 1100 and/or the system 1140 may be fully automated for standardized implementation across multiple clinical sites. Further, modifications in terms of the apparatus 1100 and/or the system 1140 and/or processing of samples may be carried out so as to increase the log total WBC depletion from 2.3 log to 4 log, while maintaining the current high recovery, which may be considered to be close to ideal for CTC isolation platforms.

### Example 2

Various embodiments may provide a negative enrichment approach for isolation of circulating tumor cells (CTCs), integrating WBC depletion and chemical-free RBC depletion in the same setup without the need for centrifugation, washing or multiple sample handling steps. Various embodiments may employ an approach based on the method 700 of FIG. 7.

Various embodiments may provide a two-step process combining WBC depletion and chemical-free RBC depletion, directly from approximately 2 mL of whole blood. In this approach, about 2 mL of whole blood may be conjugated with anti-CD45 antibodies and magnetic particles in a syringe barrel, which may then be placed inside a permanent magnet. The end of the syringe barrel may be connected to a filter membrane holder containing a precision micro-fabricated slit membrane via a luer connector. Upon activation of the valve at the end of the syringe barrel, WBC-depleted blood may flow through the precision membrane, which may selectively allow passage of RBCs and retention of nucleated cells. Thus in a single step with minimal sample handling, WBCs, RBCs and platelets may be depleted effectively.

The approach may achieve an average of >90% recovery of spiked tumor cells (e.g. an average tumor cells isolation efficiency of 94%) and >99% total WBC depletion in whole blood, with 2.25 log₁₀ enrichment, across multiple cell lines, in a simple and easy-to-use assay. The process may be completed in 1 hour, in 2 steps, without any manual sample handling. As will be described later, the results obtained and the approach of various embodiments aim to fulfill the need for a highly reliable, unbiased, standardized, and optimized CTC isolation platform, using component technologies that are validated for cell isolation.

Immunomagnetic Separation

White blood cells (WBCs) are CD45-positive and may be depleted by means of immunomagnetic approach. EasySep Human Whole Blood CD45 Depletion Kit (StemCell Technologies, Canada) was incorporated in the experiments. The kit consists of two-part components: Anti-CD45 Tetrameric Antibody Complexes (TAC) and EasySep® Magnetic Nanoparticles. This kit was used for immunomagnetic WBCs depletion during the sample preparation in this example.

Sample Preparation

Blood samples taken from healthy donors were used to spike the cancer cell lines for conducting the experiments. BD Vacutainer® K2 EDTA (Catalog#367863, BD) blood tubes were used for venipuncture collection. EDTA acts as an anticoagulant that prevents the blood clotting. The EDTA tubes did not contain any preservative or fixative agents. Approximately 2 ml of blood was diluted with about 2 ml of dilution buffer in a 5-mL syringe barrel (Catlog#302135, BD) for each experiment. Approximately 100 μl of Anti-CD45 TAC (Catlog#18289, STEMCELL Technologies) was conjugated with approximately 100 μl of magnetic nanoparticles (Catlog#18289, STEMCELL Technologies) and incubated for about 10 min. Then, the conjugated mixture was added to the diluted blood and incubated for about 30 min. “The Big Easy” EasySep® Magnet (Catlog#18001, STEMCELL Technologies) was placed around the syringe barrel to separate the labeled cells, mainly WBCs for about 15 min. After incubation, the sample was passed through a micro slit membrane for red blood cell (RBC) depletion. WBC depletion was determined by counting the cells before and after the immunomagnetic depletion procedure using an automatic haematology analyzer (Horiba Micro ES60, Horiba).

Micro Slit Membrane Fabrication

Micro slit membranes were fabricated using Parylene-C. A 25 mm diameter circular membrane includes periodically arranged, precision etched slits with slit dimension of about 5.5 μm in width and about 40 μm in length. The choice of 25 mm membrane was to allow for high throughput blood flow with least fluidic resistance. It is important to minimize the fluidic resistance (pressure drop across the membrane), as it may affect the viability and morphology of the cells and may potentially force target cells to pass through. The 25 mm membrane was also designed to be compatible with a doubly-supported, commercial filter holder (Catlog#F0102-BA, SPI Supplies), thus reducing overhead manufacturing cost of fixture as well as creating a stable and repeatable experimental setup. In this non-limiting example, the objective is to capture as many nucleated cells as possible on the membrane, including WBCs, in contrast to conventional approaches involving slit filters which used higher pressures to force WBCs and RBCs through the membrane. Thus minimal pressure was applied to drive blood, which may lead to well-preserved viability and morphology of isolated cells.

The fabrication of RBC elimination slit-membrane was performed in-house. The above-mentioned design was preferred over other micro filter designs for cell separation due to its unique advantages in retaining PBMCs at a high flow rate, while eliminating RBCs.

A silicon (Si) wafer deposited with about 1 μm thick silicon dioxide (SiO₂) by using plasma-enhanced chemical vapour deposition (PECVD) was used as a substrate. A monolayer of adhesion promoter, gamma-Methacryloxypropyltrimethoxysialne (A-174 silane) was functionalized on the substrate to promote subsequent poly-p-xylylene (parylene-C) deposition. About 10 μm-thick Parylene-C was deposited by Parylene Deposition System (PDS 2010 Labcoter 2, Specialty Coating System, Inc). A layer of approximately 100 nm thick Chromium (Cr), serving as a hard mask, was deposited on top of the Parylene-C film via electron beam evaporation. With assistance of photolithography, a thin Cr layer was defined by CEP-200 Chrome etchant with an optimum etching time of about 50 seconds. Subsequently, micro slits were developed on Parylene-C membrane via reactive ion etching (RIE). Finally, the filter membrane patterned with rectangular micro slits was released from the silicon dioxide substrate by using buffered oxide etchant (BOE) or hydrogen fluoride (HF) vapour.

This non-limiting example of the membrane design may maximize the depletion of platelets, RBCs, and smaller-sized leukocytes such as lymphocytes, monocytes, and granulocytes that may escape from upstream immunomagnetic depletion, while maximizing retention of other nucleated cells.

The rectangular slit or elongate pore, as opposed to the commonly used circular pore design, also helps to alleviate the pressure build up on the cells, as the cells may not fully occupy the porous structure. RBCs, being 1000-times more deformable, may easily re-orient themselves to pass through the opening while nucleated cells may not pass through easily. Hence, the rectangular slit design may maximize the flow rate while minimizing the required pressure to drive the flow. This may preserve the viability and morphology of target cells.

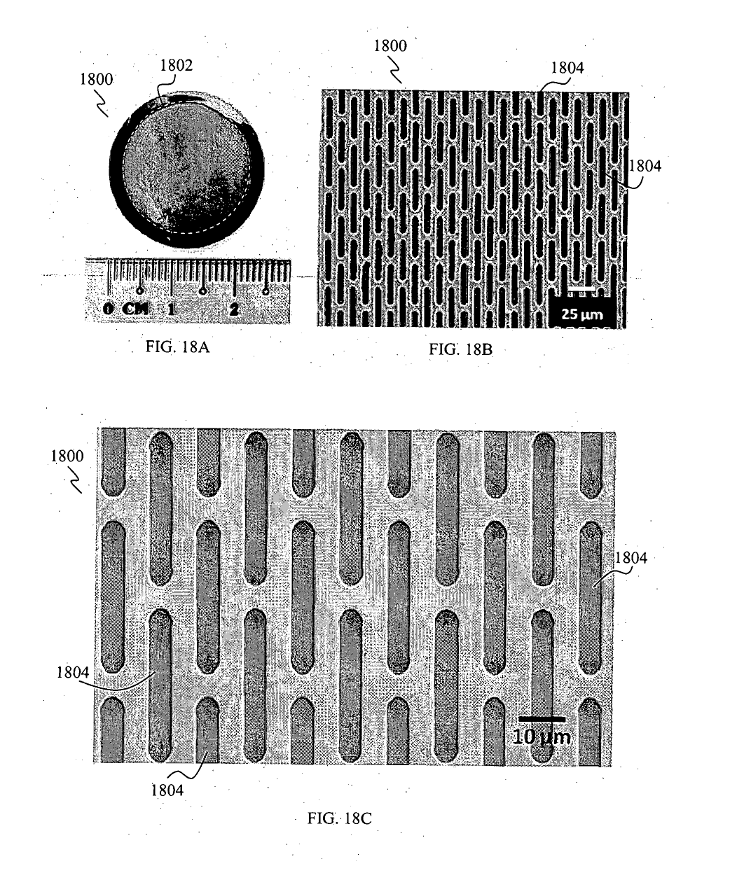

FIGS. 18A to 18C show photographs illustrating different views of a Parylene micro slit membrane 1800, according to various embodiments. FIG. 18A shows an overview picture of the membrane 1800 taken by digital camera, FIG. 18B shows an image taken at 20× using optical microscope, and FIG. 18C shows an image taken at 50X using optical microscope. Parylene was employed as the membrane material because it possesses high bio-compatibility, excellent mechanical properties with Young's modulus (about 4 GPa) and an inert chemical property, which may be resistant to moisture and most chemicals. In addition, Parylene deposition may be conformal and may yield membranes of uniform thickness.

The membrane 1800 has an active diameter of about 21 mm, as represented within the dotted circle 1802, and a thickness of about 10 μm, and containing slits, as represented by 1804 for some slits, with slit dimension approximately 5.5×40 μm, and a fill factor of approximately 39%.

Spiking of Cancer Cells

Two types of cancer cell lines were used, being green florescent protein (GFP)-tagged MCF-7 human breast cancer cells (Cell Biolabs, USA) and GFP-tagged A549 lung cancer cells (Cell Biolabs, USA). These are stable cell lines with reporter protein that exhibited bright green fluorescence when exposed to blue excitation light. The MCF-7 may be useful for breast cancer studies because it has retained several ideal characteristics that are particular to the mammary epithelium. The A549 cell line was derived from the human alveolar basal epithelial cells and was used for studying lung cancer. The MCF-7 cells in this example were bigger than the A549 cells with average cell sizes of approximately 17.6 μm and 13.6 μm, respectively. These cell lines were chosen to test the robustness and applicability of the system, as will be described later, to a wider range of cancer types.

Cancer cell lines were spiked into whole blood from healthy donor before any blood processing procedures in order to simulate the actual screening of cancer patient blood. Cultured cancer cell lines were harvested and resuspended in 1X Phosphate-Buffered Saline (PBS) solution (Catlog#10010, Invitrogen) with a concentration of about 1 million cells per mL. The cells were stained with trypan blue to check for the cell count, average size and viability using an automated cell counter (Luna, Logos Biosystems). Lower concentration of cells was obtained by serial dilutions using 1×PBS. In addition, manual haemocytometer was employed in order to obtain the actual number of cells within a sample volume of about 10 μL. In the end, about 20 μL of cells was added to about 2 mL of whole blood to prepare a sample spiked with approximately 200±10 cells.

Experimental Setup

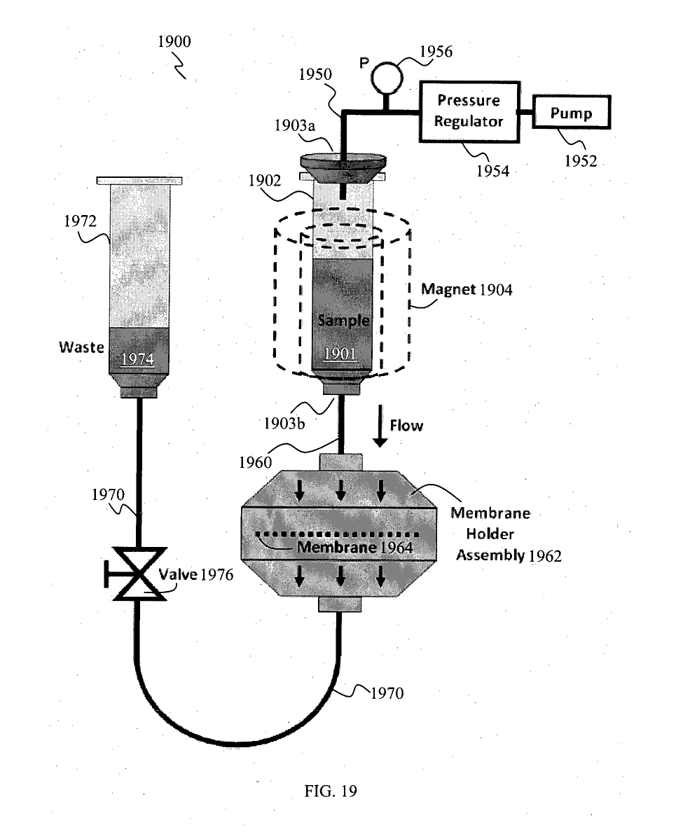

FIG. 19 illustrates a circulating tumor cell (CTC) isolation system or apparatus 1900, according to various embodiments. The apparatus 1900 includes a syringe barrel 1902, e.g. a 5-ml syringe barrel, with an input 1903a and an output 1903b, and containing blood sample 1901 conjugated with TAC and magnetic particle complex, placed within a magnet 1904 for immunomagnetic WBC depletion. A tubing 1950 may be coupled to or in fluid communication with the input 1903a and coupled to a pump 1952, a pressure regulator 1954 as well as a pressure gauge 1956. A line 1960 may represent a tubing that may be coupled to or in fluid communication with the output 1903b and coupled to a membrane holder assembly 1962 including a membrane 1964 (e.g. based on the embodiment of membrane 1800 of FIGS. 18A to 18C) arranged or secured therewithin. Alternatively, the line 1960 may represent a luer connection between the syringe barrel 1902 and the membrane holder 1962. A tubing 1970 may be coupled to or in fluid communication with the membrane holder assembly 1962 and coupled to an output chamber 1972 for collecting waste 1974. A valve 1976 may be arranged in between the membrane holder assembly 1962 and the output chamber 1972. A constant air pressure of about 3.5 mBar may be applied to gently push the sample 1901 from the syringe barrel 1902 to the micro slit membrane 1964 and then to the waste 1974.

Prior to beginning of the blood processing, all of the tubings and connections were checked to ensure a leak-free setup. The experimental setup or apparatus 1900 was primed with blocking buffer solution (1×PBS containing 5% BSA) to eliminate air bubbles and to prevent any non-specific binding of cells within fluidic pathway.

Blood sample 1901 was flown with a constant air pressure source, e.g. pump 1952 (MV20 pump, Ibidi) controlled by manufacturer's software. The pressure source 1952 was measured by a calibrated external pressure gauge (Catalog#717-100G, Fluke) 1956. In addition, the applied pressure was monitored in real time by the manufacturer's software. The sample 1901 was contained in a 5-ml syringe barrel (Catlog#302135, BD) 1902 that is connected to the membrane holder 1962 via the luer connection 1960. The micro slit membrane 1964 was supported by a mesh-like structure within the holder 1962 to prevent buckling of the membrane 1964 under fluid pressure.

WBCs within the sample 1901 were depleted by immunomagnetic separation using the magnet 1904 before being flowed down to the membrane 1964 to retain CTCs. The WBC depleted blood sample was introduced to the membrane holder 1962 by applying the pneumatic pressure after opening the pinch valve 1976.

The filtration process of 4-mL assay took less than 4 minutes resulting in a high flow rate of ˜1 mL/min. Approximately 2 ml of washing buffer, 1×PBS, was introduced to clear the membrane surface. After the microfiltration process, the cells captured onto the micro-slit membrane 1964 were stained with nuclear stain by flowing through approximately 1 μL of Hoechst 33342 dye (Catlog#H1399, Invitrogen) in about 499 μL of 1×PBS and incubated for about 2 minutes. Subsequently, the micro-slit membrane 1964 was washed with about 1 mL of washing buffer to eliminate unbound counter staining and minimize the background noise. All of the reagents were introduced sequentially at a constant pressure of 3.5 mbar in order to ensure a continuous flow.

Finally, the micro-slit membrane 1964 was carefully removed from its holder 1962 and then transferred to a microscopic slide to be inspected under a fluorescent microscope at 20× magnification.

Cell Identification

The micro slit membrane 1964 was carefully removed and placed flat on an ordinary microscope slide for cell inspection. An upright microscope (BX61, Olympus) with a motorized xy stage was used for imaging and inspection of cells. Image capturing was performed by 12-bit monochrome Rolera XR fast 1394 CCD camera (QImaging, Canada). Cells expressing positive GFP and Hoechst were counted as cancer cells. U-MWU2 and U-MWIB2 filter sets were used to visualize Hoechst and GFP probes respectively. Motorized stage was controlled by a joystick to scan through the entire membrane 1964. In addition, the software (Image Pro-Plus MDA, Media Cybernetics) was employed to stitch individual frames to create a single image file for analysis of cells that were captured on the membrane 1964. Cell counting and image capturing was performed using a 20× objective lens.

Evaluation of Depletion, Recovery and Enrichment

The depletion of WBCs, cancer cell recovery and enrichment of cancer cells on the membrane were determined as follow:

$$\begin{matrix}WBC depletion ( \% ) = \frac{{W}_{i} - {W}_{f}}{{W}_{i}} \times 100 , & ( Equation 4 ) \\ Capture efficiency ( \% ) = \frac{{C}_{R}}{{C}_{S}} \times 100 , & ( Equation 5 ) \\ {log}_{10} enrichment = log ( \frac{( \frac{{C}_{R}}{{N}_{f}} )}{( \frac{{C}_{S}}{{N}_{i}} )} ) . & ( Equation 6 )\end{matrix}$$

Wᵢ is the total number of WBC in the original sample and W_{f} is the number of WBC in the sample after immunomagnetic depletion. Both Wᵢ and W_{f} were obtained by Horiba Micros ES60 automated haematology analyzer. Cₛ is the number of cancer cells spiked into the blood sample prior to experiment, and C_{R} is the number of cancer cells counted on the micro-slit retention membrane. Nᵢ is the total number of nucleated cells in the sample prior to the experiment, which is equal to Wᵢ and, N_{f} is the number of nucleated cells retained and counted on the micro slit membrane. The final counting of cells on the membrane was performed using a Matlab-based automated image processing algorithm, CellC.

Results and Discussions

Upstream Immunomagnetic WBC Depletion

Conventional WBC depletion using TAC involved RBC lysis and centrifugation that require additional chemical reagents, laboratory equipment, skilled personnel and causing potential risks of cell loss. In comparison, the optimized protocol of various embodiments may deplete WBCs directly in whole blood, thus eliminating several steps that may potentially cause cell losses.

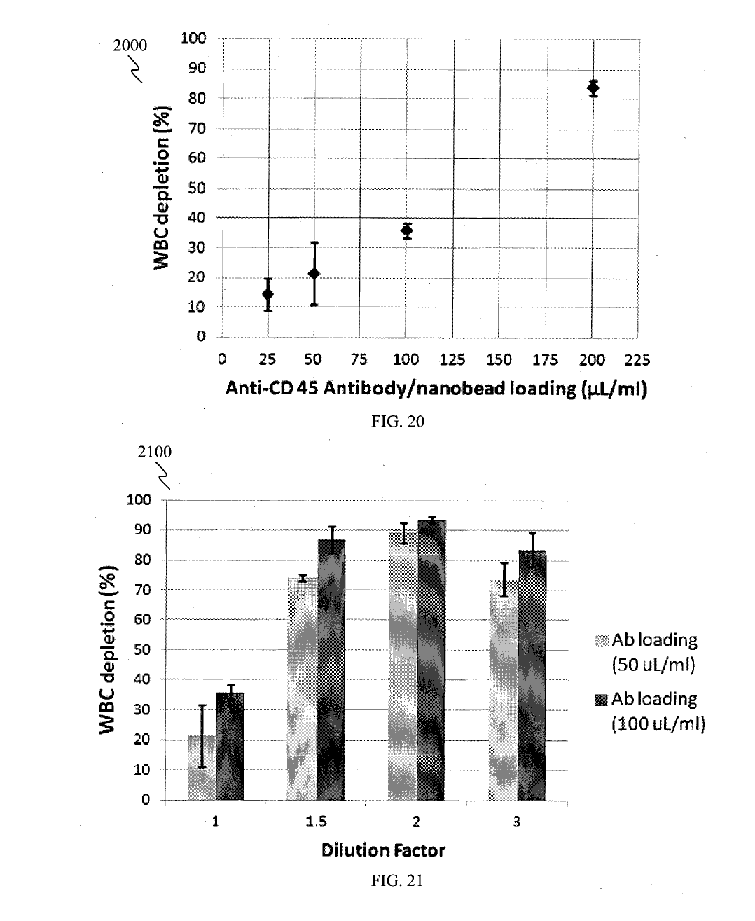

FIGS. 20 and 21 illustrate the results of the upstream WBC depletion of various embodiments. FIG. 20 shows a plot 2000 illustrating WBC depletion efficiency as a function of concentration of TAC in whole blood, showing the WBC depletion (in percentage) as a function of different amount of antibody loading into whole blood.

The effect of blood dilution on WBC depletion efficiency was also studied. FIG. 21 shows a plot 2100 of results of WBC depletion (in percentage) obtained at different dilution factors under respective antibody loadings of 50 μL/mL and 100 μL/mL, showing the WBC depletion efficiency as a function of dilution factor (0 mL, 1 mL, 2 mL and 4 mL of dilution buffer solution) and two different antibody concentrations (50 and 100 μL/mL of TAC) added to 2 mLs of blood.

Data from FIGS. 20 and 21 lead to the conclusion that the diluting ratio 1:1 of the blood sample to the dilution buffer gives an average of 90% of WBC depletion at an optimal cost and performance trade-off. Based on the analysis of 24 healthy volunteer samples, a spread of 85% and 95% WBC depletion was observed.

Optimization of CTC Isolation Process

In typical size-based isolation of CTCs, the goal is to allow passage of all normal blood cells while capturing the tumor cells, purely relying on size as the criteria. This kind of approach has many pitfalls. First, there is a significant overlap between the size and density of WBCs and CTCs. Thus the size-based systems have an inherent recovery/purity trade-off. Second, cells that are undergoing epithelial to mesenchymal transition (EMT) may assume blood-cell like characteristics, in terms of size and deformability. Such characteristics would make size-based technique highly susceptible to loss of CTCs, similar to that of epithelial cell adhesion molecule (EpCAM) based techniques. Third, the circular pore design may damage the cell morphology severely as the cells experience the blocking force of flow across the membrane. Finally, patients have varying levels of WBCs as compared to normal individuals due to their response to therapy. Due to the variation, samples from patients with high levels of WBCs demonstrate physical blockage of pores on the membrane. Hence, such instances require high pressures to clear up the clog in order for the flow to continue.

In the approach of various embodiments, without WBC depletion, it took 10 minutes and 25 mbar of pressure to run the 2-ml blood sample. Without increasing the pressure, the flow stalled. This was probably the reason why typical size-based methods used large sample dilutions, typically 10× or more. The higher pressure also may force the target cells to pass through the pores. Thus, the CTC yield may potentially be biased by the WBC content and volume of the sample.

CTC Isolation

The micro slit membrane, e.g. 1800, 1964, used for retaining CTCs was made by silicon micromachining techniques, which produced precise geometries with good reproducibility. The slits in the membrane were arranged to maximize the fill factor (e.g. 39%) without compromising the mechanical strength of the membrane. Higher fill factor coupled with large membrane area, may reduce the flow resistance, thus enabling flow at minimal pressure (3.5 mbar). RBCs have no nucleus and are highly deformable—1000 times more deformable than leukocytes. RBCs may be able to easily realign and squeeze through the slits, e.g. 1804, even at small driving pressures. The design criterion for the membrane was to maximize the nucleated cell retention, including WBCs. Because the membrane was directly coupled with upstream WBC depletion, WBC contamination was minimal in the assay of various embodiments.

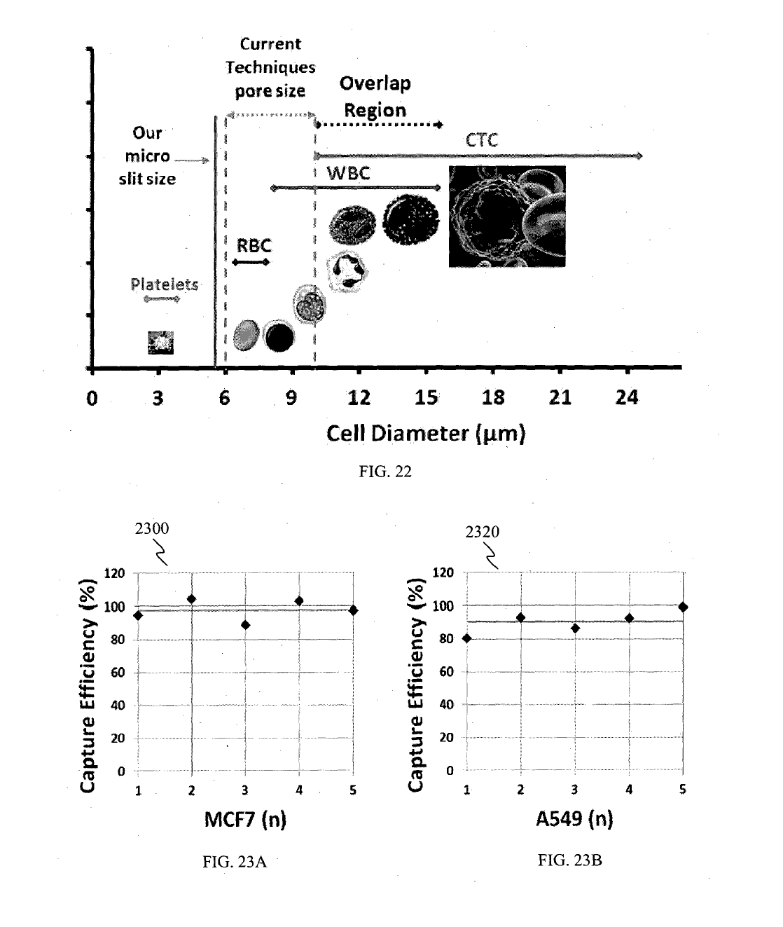

FIG. 22 shows the size ranges of a variety of cells (e.g. distribution of size of hematopoietic cells) within a blood sample compared to the sizes of tumor cells with the indication of pore size of current techniques.

A slit size (width) of approximately 5.5 μm, as employed in various embodiments, was determined to be optimal for CTC isolation, which is smaller than other current technologies. Dimensions higher than 5.5 μm may lead to loss of tumor cells due to cells wedging in the slits and undergoing deformation. Constant pressure flow was selected over constant flow rate to ensure minimal pressure on cells and to preserve their morphology and viability.

To demonstrate tumor cell detection, human whole blood spiked with GFP-tagged MCF-7 and A549 cells, upon WBC depletion was flowed through the micro slit membrane and stained with Hoechst.

FIGS. 23A and 23B show the summarized result of capture efficiency of tumor cells spiked into whole blood, illustrating results of tumor cell isolation. FIG. 23 shows a plot 2300 of the capture efficiency of GFP-tagged MCF-7 lung cancer cell line while FIG. 23B shows a plot 2320 of the capture efficiency of GFP-tagged A549 breast cancer cell line. An average capture efficiency of 97.8% for GFP-tagged MCF-7 and 90.3% for GFP-tagged A549 (n=5) was found, respectively. The choice of these two cell lines was to demonstrate the capability of the platform of various embodiments to isolate a wide range cell types with good recovery efficiency.

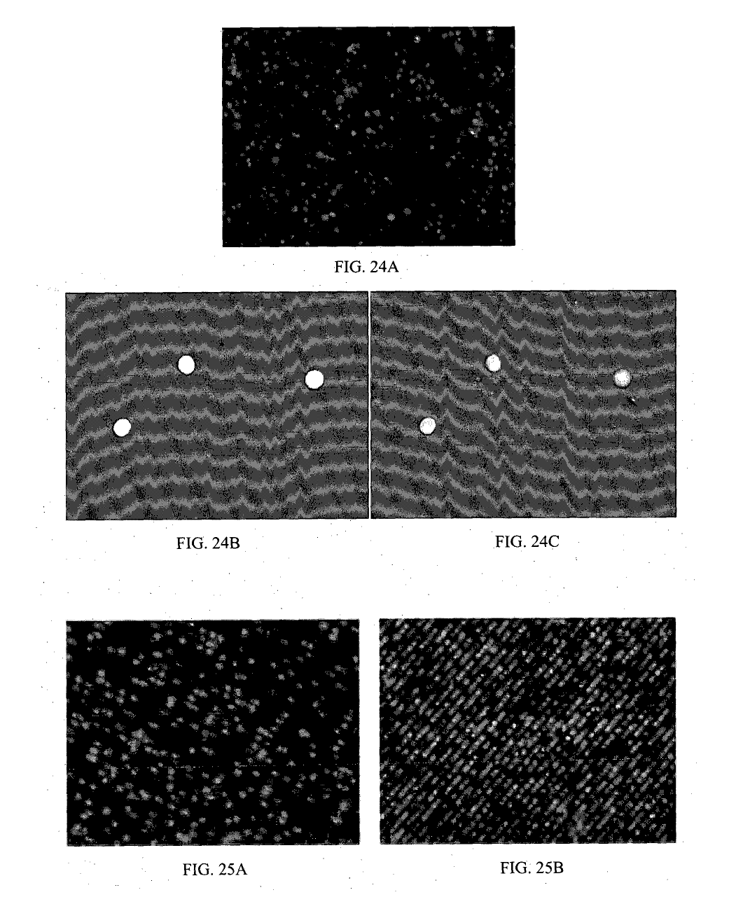

FIGS. 24A to 24C show images of MCF-7 cancer cells captured on micro slit membrane and imaged under fluorescence, respectively showing fluorescence images stained nucleated cells by Hoechst, captured GFP-tagged MCF-7 cells and merged image (of the images of FIGS. 24A and 24B) for tumor cells identification. Software (Image Pro-Plus MDA, Media Cybernetics) was used to apply the pseudo color on these cell images. It may be observed from the images that the system or apparatus 1900 may be capable of capturing tumor cells with well-preserved morphology.

Cell Recovery and Purity

The applied pressure may have critical effect on the recovery efficiency of cancer cells and depletion of WBCs. Morphology of the recovered cells may also depend heavily on the applied driving pressure. In order to achieve high CTC recovery with good cell morphology, a lower pressure may be desirable. But at lower pressure, depletion of WBCs by forcing them through porous membranes may cause more unwanted cells retained on the surface of membrane. This may subsequently lead to a lower filtration flow rate of sample, clogging, loss of target cells and/or decreased purity of the captured CTCs. This limitation may be overcome by depleting the WBCs by means of immunomagnetic separation, prior to elimination of RBCs via a slit membrane, as employed by the apparatus of various embodiments.

Since an average of 90% of WBCs were depleted upstream before the sample was flown through the membrane, a smoother and uninterrupted flow was achieved at a high flow rate of about 1 mL/min. This may enable processing a higher volume of whole blood (e.g. about 2 ml), than conventional CTC isolation assays.

FIGS. 25A and 25B show fluorescent images (Hoechst) of nucleated cells captured by micro slit membrane with and without upstream WBC depletion, respectively, showing nucleus of peripheral blood mononuclear cells (PBMCs) on membrane for experiment done with immunomagnetic WBCs depletion (255 cells) and nucleus of PBMCs on membrane for experiment done without immunomagnetic WBCs depletion (1122 cells). The effect of upstream WBC depletion may be appreciable from comparison of FIGS. 25A and 25B. The less number of WBCs may explain the smoother sample flow and higher purity of CTCs enrichment obtained using the system of various embodiments. Without pre-depletion of WBCs, it may not be possible to complete processing 2 mL of blood through the system due to clogging. Based on the number of WBCs on the membrane after sample processing, 2.25 log₁₀ enrichment over nucleated cells may be obtained.

A variety of conventional techniques are available for the isolation of CTCs with different performance parameters. A complete performance comparison between conventional methodologies is difficult due to incomplete information. Also, the analytical data is generated under highly controlled conditions. For example, 2 and 5 cells spiking experiments where the cells were handpicked and micropipette just before filtration. In another instance, spiking was performed right before the final step of processing. This type of analytical data is not fully representative of the clinical sample processing. Thus, the true comparison between technologies can only be arrived at by independent control studies.

Many sample preparation steps are typically employed for CTC isolation. Such steps include RBCs lysis, centrifugation, cells washing and resuspension, etc. These steps are necessary to improve the purity of the recovered cells to facilitate downstream molecular analysis. However, every additional step and sample handling compounds the probability of rare cell loss. Additionally, manual steps are difficult to replicate in a standardized fashion in multi-center clinical trials, raising concerns about assay standardization.

Negative enrichment based on cell size suffers from recovery/purity tradeoff, due to the size overlap between CTCs and other blood cells. In size-based filtration methods, many require a high degree of dilution in order to reduce the flux of millions of blood cells flowing through the filtration device, which may block and eventually choke the flow, thus requiring higher fluid drive pressures. Higher sample dilution ratio increases the assay footprint, prolongs assay time and potentially contributes to cell loss. Based on a conventional approach, an isolation efficiency of 83% at 2.7 log enrichment using a 7-step process involving RBC chemical lysis and centrifugation may be obtained. When generating the analytical data to calculate the cell recovery, the tumor cells were spiked after RBC depletion with lysis buffer. This overestimated the recovery of cells, as the potential cell loss due to RBC dilution was not factored-in.

As compared to conventional approach, various embodiments demonstrate a high isolation efficiency (94%) and enrichment (2.25 log₁₀) of the tumor cells in a two-step protocol directly from whole blood in an hour. As a non-limiting example, sample preparation may include approximately 2 mL blood diluted with 2 mL dilution buffer. Processing of the sample may include upstream immunomagnetic WBCs depletion integrated with CTC isolation process. As described above, CTC recovery of about 97.8% for MCF7 and about 90.3% for A549 may be achieved. In addition, the approach of various embodiments may provide a 2-step process without or with minimal manual handling, high isolation efficiency and high purity, and chemical-free RBC depletion without centrifugation.

As described above, various embodiments may address the need for a highly reliable, simple and easy-to-use assay for tumor cell isolation. A negative enrichment approach has been implemented, employing upstream immunomagnetic depletion which may be directly coupled to downstream chemical-free RBC depletion, in a simple two-step protocol. In order to prevent any loss of the target cells due to extensive sample processing and handling, such as RBCs lysis, density gradient centrifugation, cells washing and resuspension, among others, a simple yet effective protocol has been developed which includes immunomagnetic depletion of ˜90% WBCs directly from whole blood, followed by platelet and RBC depletion using a large area and high fill factor rectangular slit membrane. In various embodiments, it should be appreciated that a layer including leukocyte specific biomarkers may be coated on at least a section of an inner wall of the syringe barrel 1902, where the leukocyte specific biomarkers may also couple or bind to leukocytes (WBCs) present in the blood sample 1901.

Using the system or apparatus 1900 (FIG. 19), a high tumor cell capturing efficiency >90% across multiple cell lines has been demonstrated. The approach of various embodiments neither relies on antibodies nor size as the criteria for CTC isolation, thus, addressing the two major concerns of contemporary CTC techniques. The technique of various embodiments may yield several benefits, such as high throughput of 1 mL/min, lower fluid drive pressure of 3.5 mbar for maximal cell viability, minimal instrumentation and the ability to achieve an average of 2.25 log₁₀ enrichment in a simple 2-step protocol in approximately 1 hour.

The apparatus 1900 may be used for clinical trials to isolate CTCs from clinical patient samples. Further, modifications in terms of the apparatus 1900 and/or processing of samples may be carried out so as to achieve 4-log depletion of WBCs while maintaining >90% recovery efficiency.

As described above, various embodiments may provide a highly selective and specific meso/microfluidic, transparent fully automated enrichment system for efficient, cost effective and viable CTC enrichment. The capture of EpCAM negative, untreated and viable rare cells at an increased rate may be achieved through a combination of fully automated mesofluidic pre-enrichment and microfluidic CTC enrichment system. Thus, this approach of integrated and fully automated CTC enrichment system may substantially improve the turn-around in prognosis and diagnosis of cancer patients eliminating the cell loss due to sample transfer.

The process of various embodiments may include (a) a combination of immune-histochemical and immunomagnetic depletion in a single step using a single setup, (b) combining (a) as described above with size based depletion in a single step in the same setup, (c) enriching for CTC by a combination of (a) and (b) in a single, continuous sample flow path using a single pump.

The apparatus of various embodiments may include (a) a syringe barrel interfacing with a microfluidic chip containing an embedded precision microfabricated filter using a luer connector, (b) a syringe barrel as described in (a) surrounded by a permanent magnet for immune-depletion, (c) an entire setup containing (a) and (b) contained on a standard microscope slide (or coverslip), (d) in addition to or in lieu of the permanent magnet surrounding the syringe barrel, a permanent magnet may be placed on top of a microfluidic channel of the microfluidic chip to capture magnetically labeled cells, (e) the said syringe barrel containing no sliding piston so as not to dislodge the immune-captured cells on the cell walls, (f) the above setup easily pieceable on any standard microscope for observation, (g) the replaceable syringe barrel port may be used to introduce staining agents for immunohistochemical staining, fluorescent in situ hybridization (FISH) analysis and other biological observations of captured tumor cells, (h) the barrel inlet port may be used to introduce chemical agents that may lyse the CTCs to release DNA for further molecular analysis, (i) the microfluidic chip may be enabled with a piezoelectric substrate attached to lyse the cells by mechanical vibrations for further molecular analysis.

Various embodiments may provide a fully automated, high efficiency approach that may accomplish a combination of, some of or all of the above-mentioned processes and methods in a single, efficient and automated set-up. The approach of various to embodiments may, first, selectively deplete most of the WBCs (major contaminants) based on two methods of immune-depletion combined in a single step, followed by size based depletion of RBCs. These may be accomplished in a single step using an automated manner in a device that fits the footprint of a standard microscope slide. In contrast, conventionally, practitioners who wish to enrich CTCs by depleting other cells use various types of filter membranes. However, such method suffers from loss of CTCs, and high level of contamination from WBCs. Similarly, other practitioners who wish to enrich for CTCs by immunomagnetic enrichment, coat various types of surfaces with antibodies specific to cancer cells. However, the sensitivity and yield from these methods have been shown to be poor.

Various embodiments may be used for rare cell enrichment, detection and analysis from body fluids/tissue samples for diagnosis and therapy monitoring purposes, as well as CTCs for cancer diagnostics.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. An apparatus for separating a biological entity from a sample volume, the apparatus comprising:
an input chamber comprising:
an inlet configured to receive the sample volume; and
an outlet;
at least one magnetic element adjacent a portion of the input chamber, the magnetic element configured to provide a magnetic field in a vicinity of the portion of the input chamber to trap at least some leukocytes from the sample volume; and
a filter in fluid communication with the outlet, the filter configured to separate the biological entity.

2. The apparatus according to claim 1, wherein the input chamber further comprises a layer comprising leukocyte specific biomarkers coated on at least a section of an inner wall of the input chamber, the leukocyte specific biomarkers configured to couple to leukocytes from the sample volume.

3. The apparatus according to claim 2, wherein the layer further comprises an azide.

4. (canceled)

5. The apparatus according to claim 1, wherein the magnetic element is arranged to at least substantially surround the portion of the input chamber.

6. The apparatus according to claim 1, further comprising a plurality of magnetic elements arranged along a length of the input chamber.

7. The apparatus according to claim 1, wherein the input chamber and the filter form a closed pathway for the sample volume.

8. The apparatus according to claim 1, wherein the filter is comprised in a microfluidic device.

9. The apparatus according to claim 8, wherein the outlet of the input chamber is coupled to the filter via at least one microchannel.

10. The apparatus according to claim 9, further comprising at least one further magnetic element adjacent a portion of the microchannel, the magnetic element configured to provide a magnetic field in a vicinity of the portion of the microchannel.

11. (canceled)

12. The apparatus according to claim 8, wherein the microfluidic device comprises a piezoelectric substrate.

13. The apparatus according to claim 1, wherein the input chamber further comprises a plurality of magnetic beads couplable to leukocyte specific biomarkers configured to couple to the leukocytes from the sample volume.

14. (canceled)

15. (canceled)

16. The apparatus according to claim 1, wherein the filter comprises a single porous layer comprising a plurality of pores, each of the plurality of pores having a dimension between about 0.5 μm and about 30 μm.

17. The apparatus according to claim 1, wherein the filter comprises:
a first porous layer and a second porous layer arranged one over the other,
wherein the first porous layer comprises a plurality of first pores defined through the first porous layer,
wherein the second porous layer comprises a plurality of second pores defined through the second porous layer,
wherein one or more respective second pores are arranged to at least substantially overlap with each respective first pore such that a respective opening defined between a perimeter of the each respective first pore and a perimeter of each of the one or more respective second pores is smaller than a diameter of each first pore.

18. The apparatus according to claim 17, wherein each second pore has a diameter that is smaller than the diameter of each first pore.

19. The apparatus according to claim 18, wherein the one or more respective second pores are arranged to be within the perimeter of each respective first pore.

20. The apparatus according to claim 1, wherein the filter comprises:
a plurality of first channels arranged in a first row; and
a plurality of second channels arranged in a second row adjacent to the first row,
wherein one or more respective second channels are arranged to at least substantially overlap with each respective first channel such that a respective opening defined between an edge of the each respective first channel and an edge of each of the one or more respective second channels is smaller than a width of each first channel.

21. The apparatus according to claim 20, wherein the filter further comprises:
a plurality of third channels arranged in a third row,
wherein the second row is arranged between the first row and the third row, and
wherein one or more respective third channels are arranged to at least substantially overlap with each respective second channel such that a respective opening defined between an edge of the each respective second channel and an edge of each of the one or more respective third channels is smaller than a width of each second channel.

22. (canceled)

23. (canceled)

24. The apparatus according to claim 1, wherein the input chamber is a syringe or a vacutainer.

25-28. (canceled)

29. A method for separating a biological entity from a sample volume, the method comprising:
supplying the sample volume to an input chamber;
supplying a plurality of magnetic beads to the input chamber, the plurality of magnetic beads couplable to leukocyte specific biomarkers;
trapping leukocytes from the sample volume that are coupled to the plurality of magnetic beads at a portion of the input chamber via at least one magnetic element; and
filtering the sample volume by means of a filter for separating the biological entity.

30-32. (canceled)

33. A method for separating a biological entity from a sample volume, the method comprising:
supplying the sample volume to an input chamber;
filtering the sample volume by means of a filter for trapping the biological entity and at least some of leukocytes, fetal cells or stem cells on the filter;
supplying a plurality of magnetic beads couplable to leukocyte specific biomarkers to the filter;
flowing the trapped contents of the filter into the input chamber;
trapping the leukocytes from the sample volume that are coupled to the plurality of magnetic beads at a portion of the input chamber via at least one magnetic element; and
filtering the sample volume by means of the filter for separating the biological entity.

34. (canceled)
